SEQUENCE LISTING (28 sequences)

SEQ ID NO: 1 - protein, 17 aa, Artificial Sequence
  Description of Artificial Sequence Synthetic peptide
ISQAVHAAHAEINEAGR

SEQ ID NO: 2 - protein, 21 aa, Artificial Sequence
  Description of Artificial Sequence Synthetic peptide
MEVGWYRSPFSRVVHLYRNGK

SEQ ID NO: 3 - protein, 15 aa, Artificial Sequence
  Description of Artificial Sequence Synthetic peptide
SHLVEALYLVCGEEG

SEQ ID NO: 4 - protein, 10 aa, Artificial Sequence
  Description of Artificial Sequence Synthetic peptide
SRLGLWVRME

SEQ ID NO: 5 - protein, 11 aa, Artificial Sequence
  Description of Artificial Sequence Synthetic peptide
LQTLALWSRMD

SEQ ID NO: 6 - protein, 24 aa, Artificial Sequence
  Description of Artificial Sequence Synthetic peptide
ECCARCLVGAPFASLVATGLCFFG

SEQ ID NO: 7 - protein, 21 aa, Artificial Sequence
  Description of Artificial Sequence Synthetic peptide
LLLAEGFYTTGAVRQIFGDYK

SEQ ID NO: 8 - protein, 32 aa, Artificial Sequence
  Description of Artificial Sequence Synthetic peptide
VYIYFNTWTTCQSIAFPSKTSASIGSLCADAR

SEQ ID NO: 9 - protein, 25 aa, Artificial Sequence
  Description of Artificial Sequence Synthetic peptide
QMTFHLFIAAFVGAAATLVSLLTFM

SEQ ID NO: 10 - protein, 21 aa, Artificial Sequence
  Description of Artificial Sequence Synthetic peptide
RPSQRSKYLATASTMDHARHG

SEQ ID NO: 11 - protein, 21 aa, Artificial Sequence
  Description of Artificial Sequence Synthetic peptide
KQHYLLYIWQGSQASQDEIAA

SEQ ID NO: 12 - protein, 27 aa, Artificial Sequence
  Description of Artificial Sequence Synthetic peptide
MSPKVDVFTANTSLSSGPLPTFPLEQL

SEQ ID NO: 13 - protein, 25 aa, Artificial Sequence
  Description of Artificial Sequence Synthetic peptide
STEDFTRALGMTPAAFSALPRWKQQ

SEQ ID NO: 14 - protein, 20 aa, Artificial Sequence
  Description of Artificial Sequence Synthetic peptide
VKGESLFHSSKSMDLRVNGE

SEQ ID NO: 15 - DNA, 51 bp, Artificial Sequence
  Description of Artificial Sequence Synthetic nucleotide
atttcccaggctgttcatgccgcacatgctgagatcaatgaggcaggacgt

SEQ ID NO: 16 - DNA, 63 bp, Artificial Sequence
  Description of Artificial Sequence Synthetic nucleotide
atggaagtcggttggtatcgttcccctttttcacgtgtggtgcatctttaccgcaacgggaaa

SEQ ID NO: 17 - DNA, 45 bp, Artificial Sequence
  Description of Artificial Sequence Synthetic nucleotide
agccacttagtcgaagccctttacctggtttgcggggaagagggt

SEQ ID NO: 18 - DNA, 30 bp, Artificial Sequence
  Description of Artificial Sequence Synthetic nucleotide
tctcgcttgggattatgggttcgtatggaa

SEQ ID NO: 19 - DNA, 33 bp, Artificial Sequence
  Description of Artificial Sequence Synthetic nucleotide
ttgcaaaccttggcgctgtggtcgcgcatggat

SEQ ID NO: 20 - DNA, 72 bp, Artificial Sequence
  Description of Artificial Sequence Synthetic nucleotide
gagtgttgcgcccgttgcttagttggcgctcctttcgcgtcattggtagccacgggtttgtgcttctttggc

SEQ ID NO: 21 - DNA, 63 bp, Artificial Sequence
  Description of Artificial Sequence Synthetic nucleotide
ttacttttagcggagggcttttacacaacgggtgccgttcgtcagatcttcggtgactataaa

SEQ ID NO: 22 - DNA, 96 bp, Artificial Sequence
  Description of Artificial Sequence Synthetic nucleotide
gtatacatctacttcaacacgtggacgacgtgtcaatcgatcgcctttccgtcgaagacttcagcctctattggaagcctgtgcgctgacgcccgc

SEQ ID NO: 23 - DNA, 75 bp, Artificial Sequence
  Description of Artificial Sequence Synthetic nucleotide
cagatgacctttcacctgttcatcgcggcttttgtcggagcggctgccaccttagtcagtttattaacatttatg

SEQ ID NO: 24 - DNA, 63 bp, Artificial Sequence
  Description of Artificial Sequence Synthetic nucleotide
cgccccagccagcgctcgaaatatctggccacagcctcaacaatggatcatgctcgccacgga

SEQ ID NO: 25 - DNA, 63 bp, Artificial Sequence
  Description of Artificial Sequence Synthetic nucleotide
aaacagcactacctgttatacatctggcaaggctcccaggcttctcaagatgaaattgctgct

SEQ ID NO: 26 - DNA, 81 bp, Artificial Sequence
  Description of Artificial Sequence Synthetic nucleotide
atgagtcccaaagttgatgtttttactgcaaatacctcccttagttcgggacctttaccaacttttcccttggaacagttg

SEQ ID NO: 27 - DNA, 75 bp, Artificial Sequence
  Description of Artificial Sequence Synthetic nucleotide
tccactgaggatttcacacgcgcccttggtatgaccccagcagccttctctgctttgccacgttggaagcaacag

SEQ ID NO: 28 - DNA, 60 bp, Artificial Sequence
  Description of Artificial Sequence Synthetic nucleotide
gtaaaaggcgaatcccttttccacagctctaagtcgatggatcttcgtgtgaatggagaa

(19) United States
(12) Patent Application Publication
(10) Pub. No.: US 2022/0362358 A1
(43) Pub. Date: Nov. 17, 2022

(54) BACTERIA-ENGINEERED TO ELICIT ANTIGEN-SPECIFIC T-CELLS

(71) Applicants: Chan Zuckerberg Biohub, Inc., San Francisco, CA (US); The Board of Trustees of the Leland Stanford Junior University, Stanford, CA (US)

(72) Inventors: Michael A. Fischbach, Redwood City, CA (US); Kazuki Nagashima, Palo Alto, CA (US); Yiyin E. Chen, Stanford, CA (US)

(21) Appl. No.: 17/620,633

(22) PCT Filed: Jun. 18, 2020
(86) PCT No.: PCT/US20/38526

§ 371 Date: Dec. 17, 2021

Related U.S. Application Data
(60) Provisional application No. 63/033,811, filed on Jun. 2, 2020.
(60) Provisional application No. 62/863,594, filed on Jun. 19, 2019.

(51) Int. Cl. A61K 39/00; C12N 15/62; C12N 15/74; C12N 1/20; C12N 5/0783; A61P 37/06

(52) U.S. Cl. CPC A61K 39/0008; C12N 15/62; C12N 15/74; C12N 1/20; C12N 5/0636; A61K 39/001191; A61K 39/001198; A61K 39/001186; A61K 39/001188; A61K 39/001156; A61P 37/06; C12N 2800/101; C12N 2502/99; C12N 2502/1121; A61K 2039/523; A61K 2039/54

(57) ABSTRACT

Provided are modified microorganisms, such as live recombinant commensal bacteria, that express a heterologous antigen, and methods of using the modified microorganisms to induce an antigen-specific immune response to the heterologous antigen. The modified microorganism can be used to induce a regulatory T cell immune response to the heterologous antigen to treat an autoimmune disease in a subject in need thereof, or can be used to induce an effector T cell immune response to the heterologous antigen to treat a proliferative disease in a subject in need thereof.

### CROSS-REFERENCE TO RELATED APPLICATIONS

This application claims the benefit of and priority to U.S. Provisional Patent Application No. 62/863,594, filed Jun. 19, 2019, and U.S. Provisional Patent Application No. 63/033,811, filed Jun. 2, 2020, the disclosures of which are hereby incorporated by reference in their entirety for all purposes.

### STATEMENT AS TO RIGHTS TO INVENTIONS MADE UNDER FEDERALLY SPONSORED RESEARCH AND DEVELOPMENT

This invention was made with Government support under Grant No: DK113598 awarded by the National Institutes of Health (NIH). The Government has certain rights in the invention.

### SEQUENCE LISTING

The instant application contains a Sequence Listing which has been submitted electronically in ASCII format and is hereby incorporated by reference in its entirety. Said ASCII copy, created on Jun. 18, 2020, is named FBI-001WO_SL_ST25.txt and is 7,866 bytes in size.

### BACKGROUND OF THE INVENTION

Commensal microbiota reside primarily at barrier sites, such as the gastrointestinal tract, respiratory tract, urogenital tract and skin, where they functionally tune the innate and adaptive immune systems. Immune tolerance to these microbes must be established at each of these sites. In the gastrointestinal tract, a simple columnar epithelium is coated by a thick mucus layer that facilitates spatial segregation from luminal bacteria and also diminishes the immunogenicity of microbial antigens by delivering tolerogenic signals to resident dendritic cells. Innate lymphoid cells limit commensal-specific CD4⁺ T cell responses via an MHC class II-dependent mechanism and produce interleukin-22, which further promotes anatomical containment of microbes. Specialized gut-resident CD103⁺CD11b⁺ dendritic cells also play an important role in maintaining intestinal homeostasis by favoring induction of regulatory T (T_{reg}) cells over pro-inflammatory CD4⁺ subsets (see Scharschmidt T. C. et al., Immunity 2015, November 17; 43(5): 1011-1021). Interestingly, in other microbial niches such as the skin, certain commensal microbes (e.g., Staphylococcus epidermidis) have been demonstrated to selectively induce a CD8+ effector T cell response via interaction with dermal dendritic cells (see Naik S. et al., Nature 2015, 520:104-108).

T_{reg} cells play a major role in establishing and maintaining immune homeostasis in peripheral tissues, particularly at barrier sites where they stably reside. In the intestinal lamina propria, T_{reg} cells not only maintain self-tolerance but also play a crucial role in mediating tolerance to commensal organisms. A large percentage of gut-resident T_{reg} cells recognize commensal antigens, and thymically derived T_{reg} cells support tolerance to intestinal microbes. In addition, certain bacterial species expand T_{reg} cells in the lamina propria (Id.).

T_{regs} are a subset of T helper (T_{H}) cells, and are considered to be derived from the same lineage as naïve CD4 cells. T_{regs} are involved in maintaining tolerance to self-antigens, and preventing auto-immune disease. Tregs also suppress induction and proliferation of effector T cells (T_{eff}). T_{regs} produce inhibitory cytokines such as TGF-β, IL-35, and IL-10. T_{regs} express the transcription factor Foxp3. In humans, the majority of T_{reg} cells are MHC class II restricted CD4+ cells, but there is a minority population that are FoxP3+, MHC class I restricted, CD8+ cells. T_{regs} can also be divided into subsets: “natural” CD4+CD25+FoxP3+T_{reg} cells (nT_{regs}) that develop in the thymus, and “inducible” regulatory cells (iT_{regs}) which arise in the periphery. iT_{regs} are also CD4+CD25+FoxP3+, and develop from mature CD4+ T cells in the periphery (i.e. outside of the thymus). iT_{regs} can also express both RORγt and Foxp3 (see Sefik E., et al., “Individual intestinal symbionts induce a distinct population of RORgamma(+) regulatory T cells,” Science 2015; 349:993-997). Research has shown that TGF-β and retinoic acid produced by dendritic cells can stimulate naïve T cells to differentiate into T_{regs}, and that naïve T cells within the digestive tract differentiate into T_{regs} after antigen stimulation. iT_{regs} can also be induced in culture by adding TGF-β.

In contrast to T_{regs}, T effector (T_{eff}) cells generally stimulate a pro-inflammatory response upon antigen-specific T Cell receptor (TCR) activation via the expression or release of an array of membrane-bound and secreted proteins that are specialized to deal with different classes of pathogen. There are three classes of T_{eff} cell: CD8+ cytotoxic T cells, T_{H}1 cells, and T_{H}2 cells. CD8+ cytotoxic T cells recognize and kill target cells that display peptide fragments of intracellular pathogens (e.g., viruses) presented in the context of MHC class I molecules at the cell surface. CD8+ cytotoxic T cells store preformed cytotoxins in lytic granules which fuse with the membranes of infected target cells. CD8+ cytotoxic T cells additionally express Fas ligand, which induces apoptosis in Fas-expressing target cells. T_{H}1 and T_{H}2 cells both express CD4 and recognize peptide fragments degraded within intracellular vesicles and presented on the cell surface in the context of MHC class II molecules. T_{H}1 cells can activate a number of other immune cells, including macrophages and B cells, thereby promoting more efficient destruction and clearance of intracellular microorganisms. T_{H}2 cells stimulate the differentiation of B cells and promote the production of antibodies and other effector molecules of the humoral immune response.

### SUMMARY OF THE INVENTION

In one aspect, provided herein is a live, recombinant commensal bacterium, wherein the bacterium is engineered to express a non-native protein or peptide, wherein the protein or peptide is associated with a host disease or condition, wherein upon administration of the bacterium to the host resulting in colonization of a native host niche by the bacterium, the host mounts an adaptive immune response to the non-native protein or peptide, wherein the adaptive immune response is a regulatory T-cell (T_{reg}) response or an effector T-cell (T_{effector}) response. In some embodiments, the colonization of the native host niche is persistent or transient. In certain embodiments, the native host niche is transiently colonized, and colonization is for 1 day to 60 days. In certain embodiments, the native host niche is transiently colonized, and colonization is for 3.5 days to 60 days. In certain embodiments, the native host niche is transiently colonized, and colonization is for 7 days to 28 days. In some embodiments, colonization is determined by polymerase chain reaction or colony forming assay performed on a sample obtained from the host after 1 day, 3.5 days, 7 days, 14 days, 28 days, or 60 days after administration to the host. In some embodiments, the administration results in interaction of the bacterium with a native immune system partner cell. In certain embodiments, the native immune system partner cell is an antigen-presenting cell. In certain embodiments, the antigen-presenting cell is selected from the group consisting of a dendritic cell, a macrophage, a B-cell, and an intestinal epithelial cell.

In some embodiments, the native host niche is selected from the group consisting of the gastrointestinal tract, respiratory tract, urogenital tract, and skin. In some embodiments, the non-native protein or peptide is a host protein or peptide. In some embodiments, the bacterium is a Gram-negative bacterium. In certain embodiments, the Gram-negative bacterium is selected from the group consisting of Bacteroides thetaiotaomicron, Helicobacter hepaticus and Parabacteroides sp. In certain embodiments, the bacterium is a Gram-positive bacterium. In certain embodiments, the Gram-positive bacterium is selected from the group consisting of Staphylococcus epidermidis, Faecalibacterium sp. and Clostridium sp.

In some embodiments, the administration is via a route selected from the group consisting of topical, enteral, parenteral and inhalation. In certain embodiments, the administration route is topical. In some embodiments, the bacterium is S. epidermidis. In certain embodiments, the administration route is enteral. In some embodiments, the bacterium is selected from the group consisting of Bacteroides spp., Clostridium spp., Helicobacter spp., Parabacteroides spp, and Prevotella spp. In some embodiments, the bacterium is selected from the group consisting of Bacteroides thetaiotaomicron, Bacteroides vulgatus and Bacteroides finegoldii.

In some embodiments, the adaptive immune response is a T_{reg} response and the bacterium is selected from the group consisting of Bacteroides spp., Helicobacter spp., Parabacteroides spp., Clostridium spp., Staphylococcus spp., Lactobacillus spp., Fusobacterium spp., Enterococcus spp., Acenitobacter spp., Flavinofractor spp., Lachnospiraceae spp., Erysipelotrichaceae spp., Anaerostipes spp., Anaerotruncus spp., Coprococcus spp., Clostridiales spp., Odoribacter spp., Collinsella spp., Bifidobacterium spp., Streptococcus spp., and Prevotella spp. In certain embodiments, the adaptive immune response is a T_{reg} response and the bacterium is selected from the group consisting of Clostridium ramosum, Staphylococcus saprophyticus, Bacteroides thetaiotaomicron, Clostridium histolyticum, Lactobacillus rhamnosus, Parabacteroides johnsonii, Fusobacterium nucleatum, Enterococcus faecium, Lactobacillus casei, Acenitobacter lwofii, Bacteroides ovatus, Bacteroides vulgatus, Bacteroides uniformis, Bacteroides finegoldii, Clostridium spiroforme, Flavonifractor plautii, Clostridium hathewayi, Lachnospiraceae bacterium, Clostridium bolteae, Erysipelotrichaceae bacterium, Anaerostipes caccae, Anaerotruncus colihominis, Coprococcus comes, Clostridium asparagiforme, Clostridium symbiosum, Clostridium ramosum, Clostridium sp. D5, Clostridium scindens, Lachnospiraceae bacterium, Clostridiales bacterium, Bacteroides intestinalis, Bacteroides caccae, Bacteroides massiliensis, Parabacteroides distasonis, Odoribacter splanchnicus, Collinsella aerofaciens, Acinetobacter lwoffii, Bifidobacterium breve, Bacteroides finegoldii, Bacteroides fragilis, Bacteroides massiliensis, Bacteroides ovatus, Bifidobacterium bifidum, Lactobacillus acidofilus, Lactobacillus casei, Lactobacillus reuteri, Streptococcus thermophilus, and Prevotella histicola. In certain embodiments, the bacterium is selected from the group consisting of Bacteroides thetaiotaomicron, Bacteroides vulgatus, Bacteroides finegoldii and Helicobacter hepaticus.

In some embodiments, the disease or condition is an autoimmune disorder. In certain embodiments, the autoimmune disorder is selected from the group consisting of multiple sclerosis, diabetes mellitus Type I, rheumatoid arthritis, systemic lupus erythematosus, inflammatory bowel disease, celiac disease, Graves' disease, Hashimoto's autoimmune thyroiditis, vitiligo, rheumatic fever, pernicious anemia/atrophic gastritis, alopecia areata, immune thrombocytopenic purpura, temporal arteritis, ulcerative colitis, Crohn's disease, scleroderma, antiphospholipid syndrome, autoimmune hepatitis type 1, primary biliary cirrhosis, Sjogren's syndrome, Addison's disease, dermatitis herpetiformis, Kawasaki disease, sympathetic ophthalmia, HLA-B27 associated acute anterior uveitis, primary sclerosing cholangitis, discoid lupus erythematosus, polyarteritis nodosa, CREST Syndrome, myasthenia gravis, polymyositis/dermatomyositis, Still's disease, autoimmune hepatitis type 2, Wegener's granulomatosis, mixed Connective tissue disease, microscopic polyangiitis, autoimmune polyglandular syndrome, Felty's syndrome, autoimmune hemolytic anemia, chronic inflammatory demyelinating polyneuropathy, Guillain-Barre Syndrome, Behcet disease, autoimmune neutropenia, bullous pemphigoid, essential mixed cryoglobulinemia, linear morphea, autoimmune polyglandular syndrome 1 (APECED), acquired hemophilia A, Batten disease/neuronal ceroid lipofuscinoses, autoimmune pancreatitis, Hashimoto's encephalopathy, Goodpasture's disease, pemphigus vulgaris, autoimmune disseminated encephalomyelitis, relapsing polychondritis, Takayasu arteritis, Churg-Strauss syndrome, epidermolysis bullosa acquisita, cicatricial pemphigoid, pemphigus foliaceus, autoimmune hypoparathyroidism, autoimmune hypophysitis, autoimmune inner ear disease, autoimmune lymphoproliferative syndrome, autoimmune oophoritis, autoimmune orchitis, autoimmune polyglandular syndrome, Cogan's syndrome, encephalitis lethartica, erythema elevatum diutinum, Evans syndrome, immunodysregulation polyendocrinopathy enteropathy X-linked (IPEX), Issac's syndrome/acquired neuromyotonia, Miller Fisher syndrome, Morvan's syndrome, PANDAS, POEMS syndrome, Rasmussen's encephalitis, stiff-person syndrome, Vogt-Koyanagi-Harada syndrome, neuromyelitis optica, graft vs host disease, and autoimmune uveitis. In certain embodiments, the autoimmune disorder is selected from the group consisting of multiple sclerosis and diabetes mellitus Type I.

In some embodiments, the non-native protein or peptide is selected from the group consisting of ovalbumin, myelin oligodendrocyte glycoprotein, insulin, chromogranin A, hybrid insulin peptides, proteolipid protein, myelin basic protein, villin, epithelial cellular adhesion molecule, collagen alpha-1, aggrecan core protein, 60 kDa chaperonin 2, vimentin, alpha-enolase, fibrinogen alpha chain, fibrinogen beta chain, chitinase-3-like protein, 60 kDa mitochondrial heat shock protein, matrix metalloproteinase-16, thyroid peroxidase, thyrotropin receptor, thyroglobulin, gluten, TSHR protein, glutamate decarboxylase 2, receptor-type tyrosine-protein phosphatase-like N, glucose-6-phosphatase 2, insulin isoform 2, zinc transporter 8, glutamate decarboxylase 1, GAD65, UniProt:A2RGMO, integrin alpha-Iib, integrin beta-3, EBV DNA polymerase catalytic subunit, 2′3′-cyclic-nucleotide 3′ phosphodiesterase, myelin associated oligodendrocyte basic protein, small nuclear ribonucleoprotein, U1 small nuclear ribonucleoprotein, histone H2B, histone H2A, histone H3.2, beta-2-glycoprotein, histone H4, 60S ribosomal protein L7, TNF-alpha, myeloperoxidase, Cbir1, MS4A12, DNA topoisomerase, CYP2D6, O-phosphoseryl-tRNA selenium transferase, pyruvate dehydrogenase complex, spectrin alpha chain, steroid 21-hydroxylase, acetylcholine receptor, MMP-16, keratin associated proteins. Chondroitin sulfate proteoglycan 4, myeloblastin, U1 small nuclear ribonucleoprotein 70 kDa, blood group Rh(D), blood group Rh(CE), myelin P2 protein, peripheral myelin protein 22, myelin protein P0, S-arrestin, collagen Alpha-1, coagulation factor VIII, collagen alpha-3(IV), desmoglein-3, desmoglein-1, Insulin-2, major DNA-binding protein, tyrosinase, 5,6-dihydroxyindole-2-carboxylic acid oxidase, HLA-A2, aquaporin-4, myelin proteolipid protein, ABC transporter, HLA I B-27 alpha chain, HLA I B-7 alpha chain, and retinol-binding protein 3. In certain embodiments, the non-native protein or peptide is selected from the group consisting of ovalbumin, myelin oligodendrocyte glycoprotein, insulin, chromogranin A, hybrid insulin peptides, proteolipid protein, myelin basic protein, villin, epithelial cellular adhesion molecule, In some embodiments, the bacterium is engineered to secrete the expressed protein or peptide. In some embodiments, the bacterium is engineered to express a fusion protein comprising the protein or peptide and a native bacterial protein or portion thereof. In some embodiments, the protein or peptide is fused to the N-terminus or the C-terminus of the native bacterial protein or portion thereof. In some embodiments, the native bacterial protein is selected from the group consisting of sialidase, endonuclease, secreted endoglycosidase, anti-sigma factor, thiol peroxidase, hypothetical protein BT_2621, hypothetical protein BT_3223, peptidase, Icc family phosphohydrolase, exo-poly-alpha-D-galacturonosidase, and hypothetical protein BT_4428. In certain embodiments, the native bacterial protein is sialidase or anti-sigma factor.

In some embodiments, the adaptive immune response is a T_{effector} response and the bacterium is selected from the group consisting of S. epidermidis, Corynebacterium spp., Parabacteroides distasonis, Parabacteroides gordonii, Alistipes senegalensis, Parabacteroides johnsonii, Paraprevotella xylaniphila, Bacteroides dorei, Bacteroides uniformis JCM 5828, Eubacterium limosum, Ruminococcaceae bacterium cv2, Phascolarctobacterium faecium, Fusobacterium ulcerans, Klebsiella pneumoniae, Clostridium bolteae 90B3, Clostridium cf saccharolyticum K10, Clostridium symbiosum WAL-14673, Clostridium hathewayi 12489931, Ruminococcus obeum A2-162, Ruminococcus gnavus AGR2154, Butyrate-producing bacterium SSC/2, Clostridium sp. ASF356, Coprobacillus sp. D6 cont1.1, Eubacterium sp. 3_1_31 cont1.1, Erysipelotrichaceae bacterium 21_3, Subdoligranulum sp. 4_3_54A2FAA, Ruminococcus bromii L2-63, Firmicutes bacterium ASF500, Firmicutes bacterium ASF500, Bacteroides dorei 5_1_36/D4 supercont2.3, Bifidobacterium animalis subsp. Lactis ATCC 27673, and Bifidobacterium breve UCC2003. In certain embodiments, the bacterium is selected from the group consisting of S. epidermidis LM087 and Corynebacterium spp.

In some embodiments, the disease or condition is a proliferative disorder. In some embodiments, the proliferative disorder is cancer. In certain embodiments, the cancer is selected from melanoma, basal cell carcinoma, squamous cell carcinoma, and testicular cancer. In certain embodiments, the cancer is melanoma.

In some embodiments, the non-native protein or peptide is selected from the group consisting of PMEL, TRP2, MART-1, NY-ESO, MAGE-A, and a neoantigen. In certain embodiments, the non-native protein or peptide is PMEL.

In some embodiments, the bacterium is engineered to secrete the expressed protein or peptide. In some embodiments, the bacterium is engineered to express a fusion protein comprising the protein or peptide and a native bacterial protein or portion thereof. In certain embodiments, the protein or peptide is fused to the N-terminus or the C-terminus of the native bacterial protein or portion thereof. In some embodiments, the native bacterial protein is selected from the group consisting of sialidase, endonuclease, secreted endoglycosidase, anti-sigma factor, thiol peroxidase, hypothetical protein BT_2621, hypothetical protein BT_3223, peptidase, Icc family phosphohydrolase, exo-poly-alpha-D-galacturonosidase, and hypothetical protein BT_4428. In certain embodiments, the native bacterial protein is sialidase or anti-sigma factor.

In some embodiments, the bacterium is administered in combination with a high-complexity defined microbial community.

In some embodiments, the host is a mammal. In certain embodiments, the mammal is a human.

In another aspect, provided herein is a polynucleotide used to engineer the recombinant commensal bacterium disclosed herein.

In another aspect, provided herein is a method for generating an antigen-presenting cell displaying an antigen derived from a non-native protein or peptide, comprising: administering the recombinant commensal bacterium disclosed herein to a subject, wherein the administration results in colonization of the native host niche by the bacterium, internalization of the bacterium by an antigen-presenting cell, and presentation of the antigen by the antigen-presenting cell. In certain embodiments, the colonization of the native host niche is persistent or transient. In certain embodiments, the native host niche is transiently colonized, and colonization is for 1 day to 60 days. In certain embodiments, the native host niche is transiently colonized, and colonization is for 3.5 days to 60 days. In certain embodiments, the native host niche is transiently colonized, and colonization is for 7 days to 28 days. In certain embodiments, colonization is determined by polymerase chain reaction or colony forming assay performed on a sample obtained from the host after 1 day, 3.5 days, 7 days, 14 days, 28 days, or 60 days after administration to the host.

In some embodiments, the administration results in interaction of the bacterium with a native immune system partner cell. In some embodiments, the native immune system partner cell is the antigen-presenting cell. In certain embodiments, the antigen-presenting cell is selected from the group consisting of a dendritic cell, a macrophage, a B-Cell, and an intestinal epithelial cell. In certain embodiments, the native host niche is selected from the group consisting of the gastrointestinal tract, respiratory tract, urogenital tract, and skin. In some embodiments, the presentation is within an MHC II complex. In some embodiments, the presentation is within an MHC I complex. In some embodiments, the bacterium is a Gram-negative bacterium. In certain embodiments, the Gram-negative bacterium is selected from the group consisting of Bacteroides thetaiotaomicron, Helicobacter hepaticus, Parabacteroides sp., and Prevotella spp. In some embodiments, the bacterium is a Gram-positive bacterium. In certain embodiments, the Gram-positive bacterium is selected from the group consisting of Staphylococcus epidermidis, Faecalibacterium sp. and Clostridium sp.

In some embodiments, the administration is via a route selected from the group consisting of topical, enteral, parenteral and inhalation. In some embodiments, the route is topical. In certain embodiments, the bacterium is S. epidermidis. In some embodiments, the route is enteral. In certain embodiments, the bacterium is selected from the group consisting of Bacteroides spp., Clostridium spp., Helicobacter spp., Parabacteroides spp, and Prevotella spp. In certain embodiments, the bacterium is selected from the group consisting of Bacteroides thetaiotaomicron, Bacteroides vulgates and Bacteroides finegoldii. In some embodiments, the native bacterial protein is selected from the group consisting of sialidase, endonuclease, secreted endoglycosidase, anti-sigma factor, thiol peroxidase, hypothetical protein BT_2621, hypothetical protein BT_3223, peptidase, Icc family phosphohydrolase, exo-poly-alpha-D-galacturonosidase, and hypothetical protein BT_4428. In certain embodiments, the native bacterial protein is sialidase or anti-sigma factor. In certain embodiments, the non-native protein or peptide is melanocyte oligodendrocyte glycoprotein. In certain embodiments, the disease or condition is multiple sclerosis.

In another aspect, provided herein is a method for generating a T-cell response in a subject, comprising: administering the recombinant commensal bacterium disclosed herein to the subject, wherein the administration results in colonization of a native host niche by the bacterium and generation of the T-cell response, wherein the T-cell response is to an antigen derived from the non-native protein or peptide. In some embodiments, the colonization of the native host niche is persistent or transient. In certain embodiments, the native host niche is transiently colonized, and colonization is for 1 day to 60 days. In certain embodiments, the native host niche is transiently colonized, and colonization is for 3.5 days to 60 days. In certain embodiments, the native host niche is transiently colonized, and colonization is for 7 days to 28 days.

In some embodiments, colonization is determined by polymerase chain reaction or colony forming assay performed on a sample obtained from the host after 1 day, 3.5 days, 7 days, 14 days, 28 days, or 60 days after administration to the host. In some embodiments, the administration is via a route selected from the group consisting of topical, enteral, parenteral and inhalation. In certain embodiments, the route is topical. In certain embodiments, the route is enteral. In some embodiments, the bacterium is selected from the group consisting of Bacteroides spp., Clostridium spp., Helicobacter spp., Parabacteroides spp, Prevotella spp., and Staphylococcus epidermidis. In some embodiments, the T-cell response is a T_{reg} or a T_{effector} response. In certain embodiments, the route is enteral and the T-cell response is a T_{reg} response.

In some embodiments, the bacterium is selected from the group consisting of Bacteroides spp., Clostridium spp., Helicobacter spp., Parabacteroides spp, and Prevotella spp. In certain embodiments, the bacterium is selected from the group consisting of Bacteroides thetaiotaomicron, Bacteroides vulgatus, and Bacteroides finegoldii.

In some embodiments, the native bacterial protein is selected from the group consisting of sialidase, endonuclease, secreted endoglycosidase, anti-sigma factor, thiol peroxidase, hypothetical protein BT_2621, hypothetical protein BT_3223, peptidase, Icc family phosphohydrolase, exo-poly-alpha-D-galacturonosidase, and hypothetical protein BT_4428. In certain embodiments, the native bacterial protein is sialidase or anti-sigma factor. In certain embodiments, the non-native protein or peptide is myelin oligodendrocyte glycoprotein. In certain embodiments, the disease or condition is multiple sclerosis. In certain embodiments, the route is topical and the T-cell response is a T_{effector} response. In certain embodiments, the bacterium is S. epidermidis.

In another aspect, provided herein is a method of treating a disease or condition in a subject, comprising: administering the recombinant commensal bacterium disclosed herein to the subject, wherein the administration results in colonization of a native host niche by the bacterium and generation of a T-cell response, wherein the T-cell response is to an antigen derived from the non-native protein or peptide, and wherein the T-cell response treats the disease or condition in the subject.

In some embodiments, the colonization of the native host niche is persistent or transient. In certain embodiments, the native host niche is transiently colonized, and colonization is for 1 day to 60 days. In certain embodiments, the native host niche is transiently colonized, and colonization is for 3.5 days to 60 days. In certain embodiments, the native host niche is transiently colonized, and colonization is for 7 days to 28 days. In certain embodiments, colonization is determined by polymerase chain reaction or colony forming assay performed on a sample obtained from the host after 1 day, 3.5 days, 7 days, 14 days, 28 days, or 60 days after administration to the host.

In some embodiments, the disease or condition is selected from the group consisting of an autoimmune disorder and a proliferative disorder. In some embodiments, the autoimmune disorder is selected from the group consisting of multiple sclerosis, diabetes mellitus Type I, rheumatoid arthritis, systemic lupus erythematosus, inflammatory bowel disease, celiac disease, Graves' disease, Hashimoto's autoimmune thyroiditis, vitiligo, rheumatic fever, pernicious anemia/atrophic gastritis, alopecia areata, immune thrombocytopenic purpura, temporal arteritis, ulcerative colitis, Crohn's disease, scleroderma, antiphospholipid syndrome, autoimmune hepatitis type 1, primary biliary cirrhosis, Sjogren's syndrome, Addison's disease, dermatitis herpetiformis, Kawasaki disease, sympathetic ophthalmia, HLA-B27 associated acute anterior uveitis, primary sclerosing cholangitis, discoid lupus erythematosus, polyarteritis nodosa, CREST Syndrome, myasthenia gravis, polymyositis/dermatomyositis, Still's disease, autoimmune hepatitis type 2, Wegener's granulomatosis, mixed Connective tissue disease, microscopic polyangiitis, autoimmune polyglandular syndrome, Felty's syndrome, autoimmune hemolytic anemia, chronic inflammatory demyelinating polyneuropathy, Guillain-Barre Syndrome, Behcet disease, autoimmune neutropenia, bullous pemphigoid, essential mixed cryoglobulinemia, linear morphea, autoimmune polyglandular syndrome 1 (APECED), acquired hemophilia A, Batten disease/neuronal ceroid lipofuscinoses, autoimmune pancreatitis, Hashimoto's encephalopathy, Goodpasture's disease, pemphigus vulgaris, autoimmune disseminated encephalomyelitis, relapsing polychondritis, Takayasu arteritis, Churg-Strauss syndrome, epidermolysis bullosa acquisita, cicatricial pemphigoid, pemphigus foliaceus, autoimmune hypoparathyroidism, autoimmune hypophysitis, autoimmune inner ear disease, autoimmune lymphoproliferative syndrome, autoimmune oophoritis, autoimmune orchitis, autoimmune polyglandular syndrome, Cogan's syndrome, encephalitis lethartica, erythema elevatum diutinum, Evans syndrome, immunodysregulation polyendocrinopathy enteropathy X-linked (IPEX), Issac's syndrome/acquired neuromyotonia, Miller Fisher syndrome, Morvan's syndrome, PANDAS, POEMS syndrome, Rasmussen's encephalitis, stiff-person syndrome, Vogt-Koyanagi-Harada syndrome, neuromyelitis optica, graft vs host disease, and autoimmune uveitis. In certain embodiments, the autoimmune disorder is selected from the group consisting of multiple sclerosis, and diabetes mellitus Type I. In some embodiments, the proliferative disorder is cancer. In certain embodiments, the cancer is selected from melanoma, basal cell carcinoma, squamous cell carcinoma, and testicular cancer. In certain embodiments, the cancer is melanoma.

In some embodiments, the administration is via a route selected from the group consisting of topical, enteral, parenteral and inhalation. In certain embodiments, the route is topical. In certain embodiments, the bacterium is S. epidermidis. In some embodiments, the disease is cancer. In some embodiments, the cancer is melanoma. In some embodiments, the non-native protein or peptide is selected from the group consisting of a melanocyte-specific antigen and a testis cancer antigen. In some embodiments, the melanocyte-specific antigen is selected from the group consisting of PMEL, TRP2 and MART-1. In some embodiments, the testis cancer antigen is selected from the group consisting of NY-ESO and MAGE-A.

In some embodiments, the route is enteral. In some embodiments, the bacterium is selected from the group consisting of Bacteroides spp., Clostridium spp., Helicobacter spp., Parabacteroides spp, and Prevotella spp. In certain embodiments, the bacterium is selected from the group consisting of Bacteroides thetaiotaomicron, Bacteroides vulgatus, and Bacteroides finegoldii. In some embodiments, the native bacterial protein is selected from the group consisting of sialidase, endonuclease, secreted endoglycosidase, anti-sigma factor, thiol peroxidase, hypothetical protein BT_2621, hypothetical protein BT_3223, peptidase, Icc family phosphohydrolase, exo-poly-alpha-D-galacturonosidase, and hypothetical protein BT_4428. In certain embodiments, the native bacterial protein is sialidase or anti-sigma factor.

In some embodiments, the autoimmune disorder is selected from the group consisting of multiple sclerosis, diabetes mellitus Type I, rheumatoid arthritis, systemic lupus erythematosus, inflammatory bowel disease, celiac disease, Graves' disease, Hashimoto's autoimmune thyroiditis, vitiligo, rheumatic fever, pernicious anemia/atrophic gastritis, alopecia areata, immune thrombocytopenic purpura, temporal arteritis, ulcerative colitis, Crohn's disease, scleroderma, antiphospholipid syndrome, autoimmune hepatitis type 1, primary biliary cirrhosis, Sjogren's syndrome, Addison's disease, dermatitis herpetiformis, Kawasaki disease, sympathetic ophthalmia, HLA-B27 associated acute anterior uveitis, primary sclerosing cholangitis, discoid lupus erythematosus, polyarteritis nodosa, CREST Syndrome, myasthenia gravis, polymyositis/dermatomyositis, Still's disease, autoimmune hepatitis type 2, Wegener's granulomatosis, mixed Connective tissue disease, microscopic polyangiitis, autoimmune polyglandular syndrome, Felty's syndrome, autoimmune hemolytic anemia, chronic inflammatory demyelinating polyneuropathy, Guillain-Barre Syndrome, Behcet disease, autoimmune neutropenia, bullous pemphigoid, essential mixed cryoglobulinemia, linear morphea, autoimmune polyglandular syndrome 1 (APECED), acquired hemophilia A, Batten disease/neuronal ceroid lipofuscinoses, autoimmune pancreatitis, Hashimoto's encephalopathy, Goodpasture's disease, pemphigus vulgaris, autoimmune disseminated encephalomyelitis, relapsing polychondritis, Takayasu arteritis, Churg-Strauss syndrome, epidermolysis bullosa acquisita, cicatricial pemphigoid, pemphigus foliaceus, autoimmune hypoparathyroidism, autoimmune hypophysitis, autoimmune inner ear disease, autoimmune lymphoproliferative syndrome, autoimmune oophoritis, autoimmune orchitis, autoimmune polyglandular syndrome, Cogan's syndrome, encephalitis lethartica, erythema elevatum diutinum, Evans syndrome, immunodysregulation polyendocrinopathy enteropathy X-linked (IPEX), Issac's syndrome/acquired neuromyotonia, Miller Fisher syndrome, Morvan's syndrome, PANDAS, POEMS syndrome, Rasmussen's encephalitis, stiff-person syndrome, Vogt-Koyanagi-Harada syndrome, neuromyelitis optica, graft vs host disease, and autoimmune uveitis. In certain embodiments, the autoimmune disorder is multiple sclerosis. In some embodiments, the bacterium is selected from the group consisting of Bacteroides thetaiotaomicron, Bacteroides vulgatus, and Bacteroides finegoldii. In certain embodiments, the non-native protein is myelin oligodendrocyte glycoprotein.

In some embodiments, the bacterium is administered in combination with a high-complexity defined microbial community.

In some embodiments, the host is a mammal. In certain embodiments, the mammal is a human.

### BRIEF DESCRIPTION OF THE DRAWINGS

FIG. 1 illustrates an exemplary method for generating a regulatory T cell response to an exogenous antigen expressed by a recombinant bacterial strain of the disclosure.

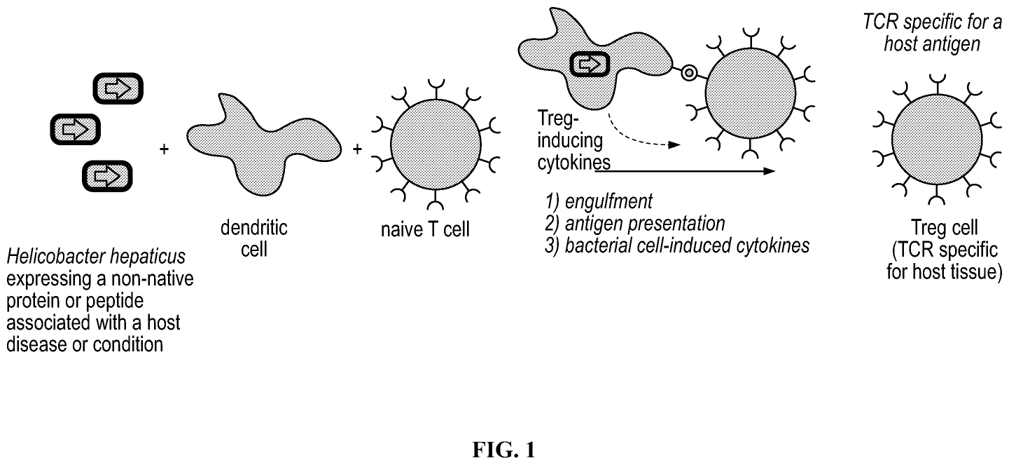

FIG. 2 shows Western blot data demonstrating expression of OVA antigen peptide by Bacteroides thetaiotaomicron engineered to express ovalbumin (OVA) peptide.

FIG. 3 shows flow cytometry analysis of OVA-specific T cells from the spleen of OTII transgenic mice co-cultured for 4 hours with B16-FLT3L stimulated DCs and OVA+B. thetaiotaomicron or WT B. thetaiotaomicron (negative control).

FIG. 4 shows Western blot data demonstrating expression of myelin oligodendrocyte glycoprotein (MOG) fusion constructs by B. thetaiotaomicron (FIG. 4A), Bacteroides vulgatus (FIG. 4B), and Bacteroides finegoldii (FIG. 4C).

FIG. 5 shows flow cytometry data of CD4+ T cell activation in in vitro co-cultures comprising antigen presenting cells (APC; splenic dendritic cells), myelin oligodendrocyte glycoprotein (MOG)-specificT cells, and live or autoclaved wild-type B. thetaiotaomicron or recombinant B. thetaiotaomicron engineered to express MOG35-55 peptide.

FIG. 6 shows Experimental Autoimmune Encephalomyelitis (EAE) scores of gnotobiotic mice administered with a mixture of B. vulgatus and B. finegoldii expressing wildtype MOG (BVF_WT) or a mixture of B. vulgatus and B. finegoldii expressing MOG fusion constructs (BVF_MOG) two weeks prior to induction of EAE (Day 0).

FIG. 7 shows flow cytometry data of CD4+ T cell populations at Day 7 in mice administered with a mixture of wild-type B. vulgatus and B. finegoldii (BVF_WT) or a mixture of recombinant B. vulgatus and B. finegoldii engineered to express MOG35-55 fusion constructs (BVF_MOG) two weeks prior to induction of EAE (Day 0).

FIG. 8 shows flow cytometry data of CD8+(FIG. 8A) and CD4+(FIG. 8B) T cell activation in in vitro co-cultures comprising APCs, ovalbumin (OVA)-specificT cells isolated from OT-I or OT-II transgenic mice, and recombinant Staphylococcus epidermidis engineered to express OVA peptide.

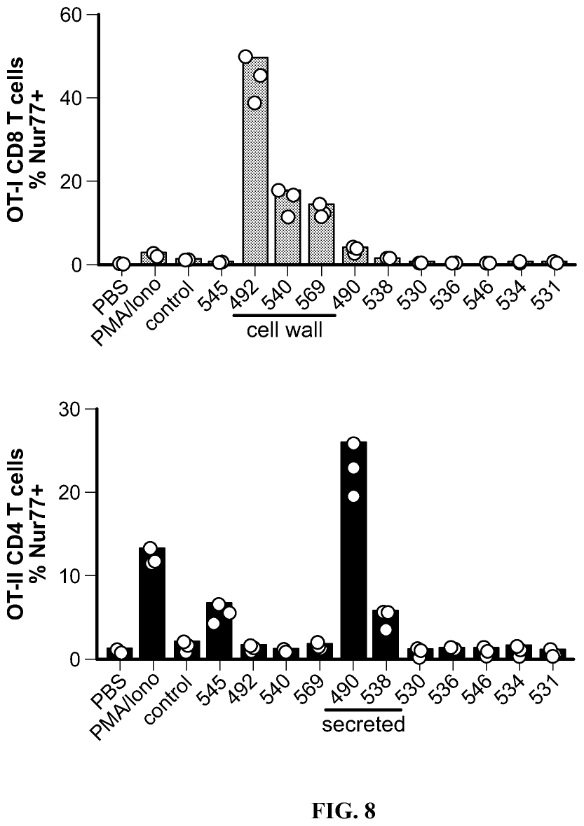

FIG. 9 shows flow cytometry data of CD8+ T cell activation in in vitro co-cultures comprising APCs, PMEL antigen-specific T cells isolated from Brest transgenic mice, and recombinant Staphylococcus epidermidis engineered to express PMEL antigen.

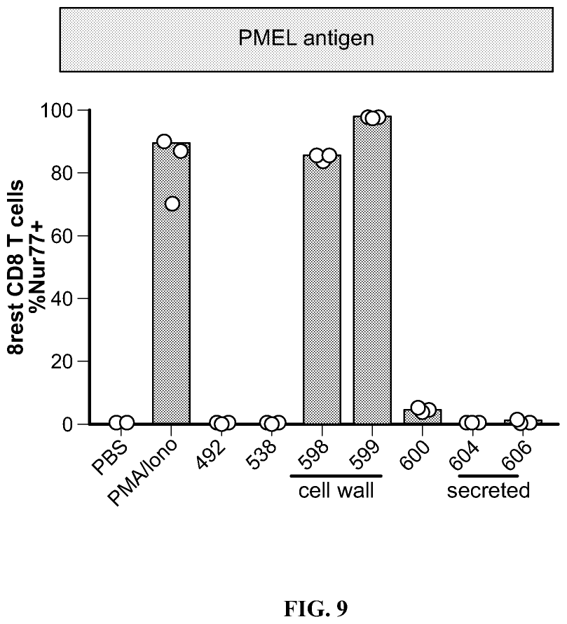

FIG. 10 shows OVA+B16F0 melanoma tumor weights (FIG. 10A) and radiance (FIGS. 10B and 10C) in mice topically administered with recombinant S. epidermidis engineered to express OVA+/− luciferase either 2 week before or 1 week after subcutaneous or intraperitoneal injection of melanoma cells.

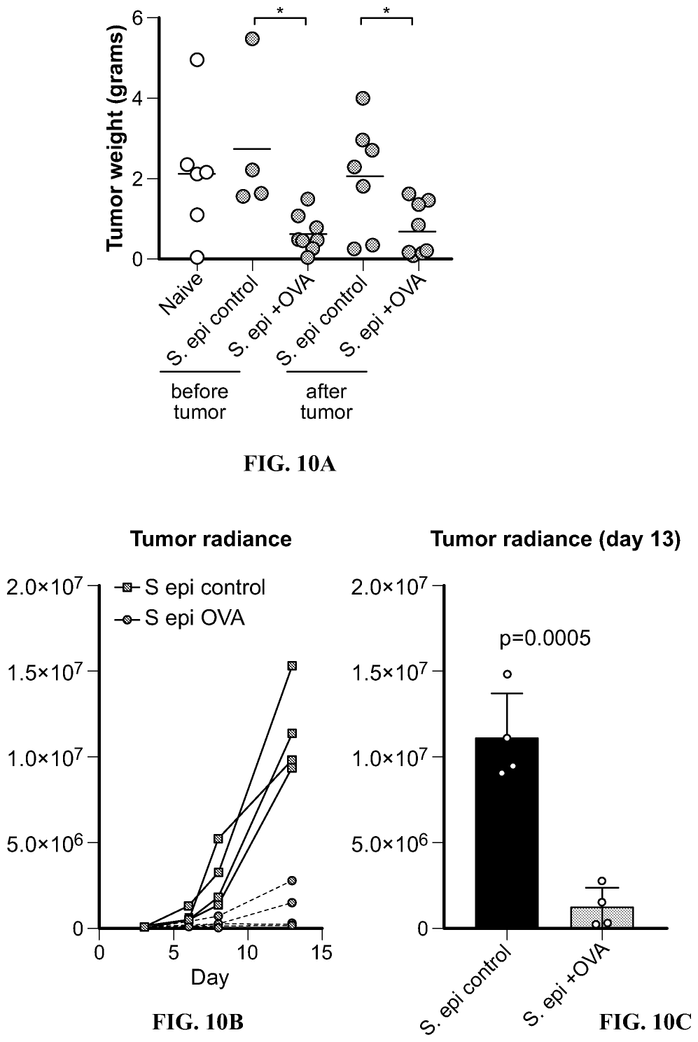

### DETAILED DESCRIPTION

### 1. Definitions

Unless defined otherwise, all technical and scientific terms used herein have the meaning commonly understood by one of ordinary skill in the art to which this disclosure belongs.

The term “a” and “an” as used herein mean “one or more” and include the plural unless the context is appropriate.

As used herein, the term “commensal” refers to a symbiotic relationship between two organisms of different species in which one derives some benefit while the other is unharmed. For example, a commensal microbe may be one that is normally present as a non-pathogenic member of a host gut microbiome, a host skin microbiome, a host mucosal microbiome, or other host niche microbiome.

As used herein, the term “bacteria” includes both singular and plural forms, such as a bacterium (single bacterial cell) and bacteria (plural), and genetically modified (recombinant) bacterial cells, bacteria and bacterial strains thereof.

As used herein, the term “commensal bacteria” refers to a bacterium, bacteria (singular or plural), bacterial cell or bacterial strain that is commensal in a vertebrate host. As will be understood by one of ordinary skill in the art, most commensal bacteria are typically symbiotic, but a commensal strain can become pathogenic or cause pathology under certain conditions, such as host immunodeficiency, microbial dysbiosis or intestinal barrier impairment. For example, a commensal bacteria is normally present as a non-pathogenic member of a host gut microbiome, a host skin microbiome, a host mucosal microbiome, or other host niche microbiome.

As used herein, the terms “colonization,” “colonized,” or “colonize” refers to the occupation of a microbe, e.g., a live, recombinant, commensal bacteria, in a niche of a host. Colonization can be persistent, e.g. lasting over 60 days, or transient, e.g. lasting between one to 60 days.

As used herein, the term “heterologous” refers to a molecule (e.g., peptide or protein) that is not normally or naturally produced or expressed by a cell or organism.

The term “antigen” refers to a molecule (e.g., peptide or protein) or immunologically active fragment thereof that is capable of eliciting an immune response. Peptide antigens are typically presented by an antigen presenting cell (APC) to an immune cell, such as a T lymphocyte (also called a T cell).

The terms “heterologous antigen,” or, in reference to proteins or peptides, “non-native,” refer to an antigen that is not normally expressed by a cell or organism. The term includes antigens, or fragments thereof, that bind to a T cell receptor and induce an immune response. For example, protein or peptide antigens are digested by antigen presenting cells (APCs) into short peptides that are expressed on the cell surface of an APC in the context of a major histocompatibility complex (MHC) class I or MHC class II molecule. Thus, the term antigen includes the peptides presented by an APC and recognized by a T cell receptor. Heterologous antigens may be host-derived antigens, or non-host derived antigens.

In reference to microbial niches in a host, the term “native” refers to an environment in or on a host in which a commensal microorganism or host immune cell is naturally present under normal, non-pathogenic conditions.

In reference to proteins expressed by a microorganism, e.g., a bacterium, the term “native” refers to a protein, or portion thereof, that is normally expressed and present in a wild-type microorganism in nature.

The term “effective amount,” or “therapeutically effective amount,” refers to an amount of a composition sufficient to prevent, decrease or eliminate one or more symptoms of a medical condition or disease when administered to a subject or patient in need of treatment.

As used herein, the term “operably linked” refers to a functional linkage between one or more nucleic acid sequences, such as between a regulatory or promoter sequence and a coding region sequence, where transcription of the coding region sequence is positively or negatively regulated by the linked regulatory sequence.

As used herein, “antigen-specific” refers to an immune response generated in a host that is specific to a given antigen. The term includes responses to antigens that are recognized by antibodies capable of binding to the antigen of interest with high affinity, and responses to antigens by T cell receptors (TCRs) that recognize and bind to a complex comprising an MHC (molecule and a short peptide that is a degradation product of the antigen of interest. Bacterial antigens are typically degraded into peptides that bind to MHC class II molecules on the surface of APCs, which are recognized by the TCR of a T cell.

As used herein, “antigen-presenting cell (APC)” refers to an immune cell that mediates a cellular immune response in a subject by processing and presenting antigens for recognition by lymphocytes such as T cells. APCs display antigen complexed with major histocompatibility complexes (MHCs) on their surfaces, often referred to as “antigen presentation.” So called “professional APCs” present antigen to helper T cells (CD4+ T cells). Examples of professional APCs include dendritic cells, macrophages, Langerhans cells and B cells.

The term “regulatory T cell” or “Treg” refers to a subpopulation of T cells that modulate the immune system, maintain tolerance to self-antigens, and prevent autoimmune disease. Tregs suppress activation, proliferation and cytokine production of CD4+ T cells and CD8+ T cells, and also suppress B cells and dendritic cells. There are two types of Treg cells. “Natural” Tregs are produced in the thymus, whereas Tregs that differentiate from naïve T cells outside the thymus (in the periphery) are called “adaptive” Tregs. Natural Tregs express the CD4 T cell receptor and CD25 (a component of the IL-2 receptor), and the transcription factor FOXP3. Tregs can also produce molecules, such as TGF-beta, IL-10 and adenosine, that suppress the immune response. Adaptive Tregs express CD4, CD45RO, Foxp3, and CD25 (see “Human CD4+CD25hi Foxp3+ regulatory T cells are derived by rapid turnover of memory populations in vivo,” Vukmanovic-Stejic M, et al., J Clin Invest. 2006 September; 116(9):2423-33).

As used herein, the terms “T effector,” “effector T,” or “T_{eff}” refer to subpopulations of T cells that exert effector functions upon cell activation, mediated by the production of membrane and secreted proteins which modulate the immune system to elicit a pro-inflammatory immune response. T_{eff} cells include CD8+ cytotoxic T cells, T_{H}1 cells, T_{H}2 cells, and T_{H}17 cells.

As used herein, the term “modified” refers to an organism, cell, or bacteria that does not exist in nature. The term is used interchangeably with “recombinant” or “engineered.”

As used herein, an “autoimmune disease” refers to a disease or pathological condition associated with or caused by the immune system attacking the body's endogenous organs, tissues, and/or cells.

As used herein, an “autoimmune antigen” refers to an antigen expressed by an endogenous organ, tissue or cell that triggers an immune response against the endogenous organ, tissue or cell.

As used herein, “animal” refers to an organism to be treated with a recombinant commensal microbe (e.g., an engineered bacterium). Animals include, but are not limited to, mammals (e.g., murines, simians, equines, bovines, porcines, canines, felines, and the like), and more preferably include humans.

As used herein, “host” refers to a non-microbial organism in or on which a commensal microorganism (e.g., a commensal bacteria) colonizes. A host can be a mammalian host, e.g, a human host.

As used herein, the terms “subject” or “patient” are used interchangeably, and refer to an organism to which a modified microorganism, e.g., a live recombinant commensal bacteria of the present invention, is administered. In some cases, a subject has an autoimmune or proliferative disease, disorder or condition. A subject can be a mammalian subject, e.g., a human subject.

As used herein, the term “pharmaceutically acceptable carrier” refers to any of the standard pharmaceutical carriers, such as phosphate buffered saline (PBS) solution, water, emulsions (e.g., such as oil/water or water/oil emulsions), and various types of wetting agents. The compositions also can include stabilizers and preservatives. For examples of carriers, stabilizers, and adjuvants, see e.g., Martin, Remington's Pharmaceutical Sciences, 15^{th} Ed. Mack Publ. Co., Easton, Pa. [1975].

### 2. Modified Microorganisms

Described herein is a modified microorganism engineered to express a heterologous antigen, and methods of inducing an immune response to the heterologous antigen in a subject. In some embodiments, the modified microorganism includes live microorganisms that colonize or are commensal in humans, such as bacteria, Archaea and fungi. In some embodiments, the live modified microorganism is a live modified bacterium, live modified bacteria or a live modified bacterial strain engineered to express a heterologous antigen. In one aspect, the modified bacteria is a commensal bacteria that expresses a heterologous antigen that is capable of inducing an antigen-specific immune response in a subject. Unlike the innate and adaptive immune response to commensal bacteria, the present disclosure provides engineered bacterial strains that express a heterologous antigen, such as a mammalian antigen. In some embodiments, the heterologous antigen is a protein or peptide that is non-native to the commensal bacterium but is native to the host. In some embodiments, the heterologous antigen is a protein or peptide that is non-native to both the commensal bacterium and the host. Because the modified bacteria are derived from a bacteria that is commensal in the host, they are not expected to be pathogenic when administered to the subject.

In some embodiments, the modified microorganism, or pharmaceutical composition comprising the modified microorganism, are administered to a native host niche. For example, a live, recombinant commensal bacterium derived from a commensal bacterium native to a host gut niche, is administered to the same host gut niche for colonization. In another example, an engineered bacterium derived from a commensal bacterium native to a host skin niche, is administered to the same host skin niche for colonization.

In some embodiments, the modified microorganism, e.g., the live, recombinant commensal bacterium, persistently colonizes a native host niche when administered to a subject. For example, in some embodiments, the live, recombinant commensal bacterium persists in the native host niche for over 60 days, over 112 days, over 178 days, over 1 year, over 2 years, or over 5 years.

In some embodiments, the modified microorganism, e.g., the live, recombinant commensal bacterium, transiently colonizes a native host niche when administered to a subject. For example, in some embodiments, the live, recombinant commensal bacterium transiently colonizes the native host niche for between 1 and 60 days, 2 and 60 days, 10 and 60 days, 20 and 60 days, 40 and 60 days, 1 and 40 days, 2 and 40 days, 10 and 40 days, 20 and 40 days, 1 and 20 days, 2 and 20 days, 10 and 20 days, 1 and 10 days, or 2 and 10 days. In some embodiments, the modified microorganism transiently colonizes the native host niche in the subject then migrates to a different niche within the host.

In some embodiments, recombinant modification of a microorganism, e.g., a live commensal bacterium, does not affect the ability of the microorganism to colonize its native host niche when administered to a subject. For example, in some embodiments, recombinant modification of a live commensal bacterium to express a non-native protein or peptide does not substantially affect the native physiology of the commensal bacterium, thereby maintaining the ability of the commensal bacterium to participate in its native synergistic interactions with the host and/or other microbial flora present in its native host niche, and facilitating the commensal bacterium's colonization of its native host niche.

The engineered bacteria are useful for inducing an antigen-specific immune response to a heterologous antigen, which results in the generation of T cells that express a T cell receptor that specifically binds to the heterologous antigen or an immunologically active fragment thereof. Thus, the engineered bacteria can be used to treat a disease or condition in a subject by administering an therapeutically effective amount of the engineered bacteria, or a pharmaceutical composition comprising the engineered bacteria, to a subject. Following administration, the subject's immune system responds by producing antigen-specific T cells that bind the heterologous antigen expressed by the bacteria. In some embodiments, the immune system responds by producing antigen-specific regulatory T cells (T_{reg}), which reduce the host's immune response against a self-antigen or other antigen corresponding to the expressed heterologous protein or peptide. In some embodiments, the immune system responds by producing antigen-specific T cells (T_{eff}), which promote an immune response against the expressed heterologous antigen, e.g. a tumor associated antigen.

The modified microorganism (e.g., bacteria, Archaea, and fungi) and methods described herein provide the advantage of generating an immune response specific for a heterologous antigen when administered to a subject. The disclosure also provides advantages over current approaches for generating antigen-specific immune cells, such as chimeric antigen receptor T cells (CAR-T cells), which are difficult and expensive to produce, are of questionable durability, and are potentially unsafe when administered to a patient because of off-target effects such as cytokine release syndrome and neurologic toxicity. In contrast, commensal microorganisms can be useful to trigger potent and long-lasting immune responses, and can be administered over the lifetime of a subject with no, or minimal, off-target effects. Live, commensal microorganisms thus provide advantages over attenuated, pathogenic non-commensal microorganisms, e.g., attenuated Listeria, which would be undesirable to administer to subjects over long time periods. Administering attenuated, pathogenic non-commensal bacteria introduces risk to a subject, especially over a long duration, due to the potential of the attenuated bacteria to revert back to a pathogenic form. In contrast, live, commensal bacteria can colonize the host subject in a non-pathogenic form for potentially long time periods, and thus provide an ongoing stimulus leading to a persistent antigen-specific T cell population, which is important since T cell responses can be short-lived.

In some embodiments, the modified microorganism is engulfed by an antigen presenting cell (APC), such as a dendritic cell, macrophage, B-cell, intestinal epithelial cell, and/or innate lymphoid cell. After being engulfed by an APC, the modified microorganism is lysed and the heterologous antigen is digested and presented to an immune cell. In some embodiments, the heterologous antigen is a protein or peptide and is digested into smaller peptide fragments, and the peptide fragments bind MHC molecules and are displayed on the surface of the APC for presentation to an immune cell. In some embodiments, the immune cell is a naïve T cell. The antigen-specific immune response can be elicited in vitro or in vivo. In some embodiments, the modified microorganism is engulfed, processed and presented by an APC to induce a T_{reg} response to the heterologous antigen. In some embodiments, the modified microorganism is engulfed, processed and presented by an APC to induce a T_{eff} response to the heterologous antigen.

### 3. Bacterial Strains

In some embodiments, the modified microorganism is a live, recombinant bacteria or bacterial strain. In some embodiments, the live, recombinant bacteria is derived from a commensal bacteria or bacterial strain. In some embodiments, the live, recombinant bacteria is derived from a commensal bacteria or bacterial strain in a mammal. In some embodiments, the live, recombinant bacteria or bacterial strain is derived from a commensal bacteria or bacterial strain in a human. In some embodiments, the live, recombinant bacteria or bacterial strain is derived from a commensal bacteria or bacterial strain native in a human niche, for example, a gastrointestinal tract, respiratory tract, urogenital tract, and/or skin.

In some embodiments, the live, recombinant bacteria is derived from a commensal bacteria that is normally non-pathogenic, for example, a bacteria that does not cause a disease, or adverse or undesired health condition, in a healthy subject that is administered the commensal bacteria (e.g., a subject having a competent immune system). In some embodiments, the live, recombinant bacteria is non-pathogenic if administered by oral, nasal, vaginal, rectal, subcutaneous, intradermal, intramuscular, or topical routes. In some embodiments, the live, recombinant bacteria is non-pathogenic if administered orally, topically or by nasal inhalation. In some embodiments, the bacteria is administered in an enteric-coated capsule.

In some embodiments, the live, recombinant bacteria is derived from a commensal bacteria that is native to the digestive tract of a mammal. For example, in some embodiments, the live, recombinant bacterium is derived from a Bacteroides spp., Clostridium spp., Faecalibacterium spp., Helicobacter spp., Parabacteroides spp., or Prevotella spp. In some embodiments, the live, recombinant bacterium is derived from Bacteroides thetaiotaomicron, Bacteroides vulgatus, Bacteroides finegoldii, or Helicobacter hepaticas.

In some embodiments, the live, recombinant bacteria is derived from a commensal bacteria that is native to the skin of a mammal. For example, in some embodiments, the live, recombinant bacterium is derived from a Staphylococcus spp., or Corynebacterium spp. In some embodiments, the live, recombinant bacterium is derived from Staphylococcus epidermidis. For example, in some embodiments, the live, recombinant bacterium is derived from S. epidermidis LM087.

In some embodiments, the live, recombinant bacteria is derived from a commensal bacteria that is Gram negative. For example, in some embodiments, the Gram negative bacteria is a Bacteroides spp., a Helicobacter spp., or a Parabacteroides spp. In some embodiments, the live, recombinant bacterium is B. thetaiotaomicron, B. vulgatus, B. finegoldii, or H. hepaticas.

In some embodiments, the live, recombinant bacteria is derived from a commensal bacteria that is Gram positive. For example, in some embodiments, the Gram positive bacteria is a Staphylococcus spp., a Faecalibacterium spp., or a Clostridium spp. In some embodiments, the live, recombinant bacterium is S. epidermidis.

In some embodiments, the live, recombinant bacteria is derived from a commensal bacteria that is known to induce a T_{reg} response in a mammalian host. For example, in some embodiments, the live, recombinant bacteria is derived from a Bacteroides spp., Helicobacter spp., Parabacteroides spp., Clostridium spp., Staphylococcus spp., Lactobacillus spp., Fusobacterium spp., Enterococcus spp., Acenitobacter spp., Flavinofractor spp., Lachnospiraceae spp., Erysipelotrichaceae spp., Anaerostipes spp., Anaerotruncus spp., Coprococcus spp., Clostridiales spp., Odoribacter spp., Collinsella spp., Bifidobacterium spp., Streptococcus spp., or Prevotella spp.

In some embodiments, the live, recombinant bacteria is derived from Clostridium ramosum, Staphylococcus saprophyticus, Bacteroides thetaiotaomicron, Clostridium histolyticum, Lactobacillus rhamnosus, Parabacteroides johnsonii, Fusobacterium nucleatum, Enterococcus faecium, Lactobacillus casei, Acenitobacter lwofii, Bacteroides ovatus, Bacteroides vulgatus, Bacteroides uniformis, Bacteroides finegoldii, Clostridium spiroforme, Flavonifractor plautii, Clostridium hathewayi, Lachnospiraceae bacterium, Clostridium bolteae, Erysipelotrichaceae bacterium, Anaerostipes caccae, Anaerotruncus colihominis, Coprococcus comes, Clostridium asparagiforme, Clostridium symbiosum, Clostridium ramosum, Clostridium sp. D5, Clostridium scindens, Lachnospiraceae bacterium, Clostridiales bacterium, Bacteroides intestinalis, Bacteroides caccae, Bacteroides massiliensis, Parabacteroides distasonis, Odoribacter splanchnicus, Collinsella aerofaciens, Acinetobacter lwoffii, Bifidobacterium breve, Bacteroides finegoldii, Bacteroides fragilis, Bacteroides massiliensis, Bacteroides ovatus, Bifidobacterium bifidum, Lactobacillus acidofilus, Lactobacillus casei, Lactobacillus reuteri, Streptococcus thermophilus, and Prevotella histicola.

In some embodiments, the live, recombinant bacteria is derived from a commensal bacteria that is known to induce a T_{eff} response in a mammalian host. For example, in some embodiments, the live, recombinant bacteria is derived from a Staphylococcus spp., Parabacteroides spp., Alistipes spp., Bacteroides spp., Eubacterium spp., Runimococcaceae spp., Phascolarctobacterium spp., Fusobacterium spp., Klebsiella spp., Clostridium spp., Coprobacillus spp., Erysipelotrichaceae spp., Subdoligranulum spp., Ruminococcus spp., Firmicutes spp., or Bifidobacterium spp.

In some embodiments, the live, recombinant bacteria is derived from S. epidermidis, Parabacteroides distasonis, Parabacteroides gordonii, Alistipes senegalensis, Parabacteroides johnsonii, Paraprevotella xylamphila, Bacteroides dorei, Bacteroides uniformis JCM 5828, Eubacterium limosum, Ruminococcaceae bacterium cv2, Phascolarctobacterium faecium, Fusobacterium ulcerans, Klebsiella pneumoniae, Clostridium bolteae 90B3, Clostridium cf. saccharolyticum K10, Clostridium symbiosum WAL-14673, Clostridium hathewayi 12489931, Ruminococcus obeum A2-162, Ruminococcus gnavus AGR2154, Butyrate producing bacterium SSC/2, Clostridium sp. ASF356, Coprobacillus sp. D6 cont1.1, Eubacterium sp. 3_1_31 cont1.1, Erysipelotrichaceae bacterium 21_3, Subdoligranulum sp. 4_3_54A2FAA, Ruminococcus bromii L2-63, Firmicutes bacterium ASF500, Firmicutes bacterium ASF500, Bacteroides dorei 5_1_36/D4 supercont2.3, Bifidobacterium animalis subsp. Lactis ATCC 27673, and Bifidobacterium breve UCC2003.

Exemplary commensal bacterial strains that can be engineered to express heterologous antigens are listed in Table 1.

TABLE 1
EXEMPLARY BACTERIAL STRAINS

| | | |
|---|---|---|
| Bacteroides | Clostridium scindens | Bacteroides dorei |
| thetaiotaomicron | | |
| Bacteroides | Lachnospiraceae | Bacteroides uniformis |
| finegoldii | bacterium | JCM 5828 |
| Bacteroides vulgatus | Clostridiales | Eubacterium limosum |
| | bacterium | |
| Helicobacter | Bacteroides | Ruminococcaceae |
| hepaticus | intestinalis | bacterium cv2 |
| Clostridium ramosum | Bacteroides caccae | Phascolarctobacterium |
| | | faecium |
| Staphylococcus | Bacteroides | Fusobacterium |
| saprophyticus | massiliensis | ulcerans |
| Clostridium | Parabacteroides | Klebsiella pneumoniae |
| histolyticum | distasonis | |
| Lactobacillus | Odoribacter | Clostridium bolteae |
| rhamnosus | splanchnicus | 90B3 |
| Parabacteroides | Collinsella | Clostridium cf. |
| johnsonii | aerofaciens | saccharolyticum K10 |
| Fusobacterium | Acinetobacter Iwoffii | Clostridium |
| nucleatum | | symbiosum WAL- |
| | | 14673 |
| Enterococcus | Bifidobacterium | Clostridium hathewayi |
| faecium | breve | 12489931 |
| Lactobacillus casei | Bacteroides fragilis | Ruminococcus obeum |
| | | A2-162 |
| Acenitobacter Iwofii | Bacteroides | Ruminococcus gnavus |
| | massiliensis | AGR2154 |
| Bacteroides ovatus | Bacteroides ovatus | Butyrate-producing |
| | | bacterium SSC/2 |
| Bacteroides | Bifidobacterium | Clostridium sp. |
| uniformis | bifidum | ASF356 |
| Clostridium | Lactobacillus | Coprobacillus sp. D6 |
| spiroforme | acidofilus | contl.l |
| Flavonifractor plautii | Lactobacillus casei | Eubacterium sp. |
| | | 3_1_31 contl.l |
| Clostridium | Lactobacillus reuteri | Erysipelotrichaceae |
| hathewayi | | bacterium 21 3 |
| Lachnospiraceae | Streptococcus | Subdoligranulum sp. |
| bacterium | thermophilus | 4 3 54A2FAA |
| Clostridium bolteae | Prevotella histicola | Ruminococcus bromii |
| | | L2-63 |
| Erysipelotrichaceae | Staphylococcus | Firmicutes bacterium |
| bacterium | epidermidis EM097 | ASF500 |
| Anaerostipes caccae | Corynebacterium | Firmicutes bacterium |
| | spp. | ASF500 |
| Anaerotruncus | Parabacteroides | Bacteroides dorei |
| colihominis | distasonis | 5_1_36/D4 |
| | | supercont2.3 |
| Coprococcus comes | Parabacteroides | Bifidobacterium |
| | gordonii | animalis subsp. Lactis |
| | | ATCC 27673 |
| Clostridium | Alistipes senegalensis | Bifidobacterium breve |
| asparagiforme | | UCC2003 |
| Clostridium | Parabacteroides | Bacteroides dorei |
| symbiosum | johnsonii | |
| Clostridium ramosum | Paraprevotella | Bacteroides uniformis |
| | xylaniphila | JCM 5828 |
| Clostridium sp. D5 | Clostridium scindens | Eubacterium limosum |

### 4. Heterologous Antigens

In some embodiments, modified microorganisms, e.g., live, recombinant commensal bacteria, are engineered to express a heterologous antigen that is not naturally expressed in a bacteria. For example, in some embodiments, the heterologous antigen normally exists in, is present in, or is expressed by a non-bacterial host. In some embodiments, the non-bacterial host is an animal that is a natural host of the commensal bacteria from which the modified microorganism is derived. In some embodiments, the heterologous antigen normally exists in, is present in or is expressed by the host of the commensal bacteria. In some embodiments, the heterologous antigen is an antigen that exists in a vertebrate or mammal. In some embodiments, the heterologous antigen is a mammalian antigen, such as a mouse or human antigen. In some embodiments, the heterologous antigen is a protein or antigenic fragment thereof.

In some embodiments, the heterologous antigen is an autoimmune antigen. For example, in some embodiments, the heterologous antigen is myelin oligodendrocyte glycoprotein, insulin, chromogranin A, hybrid insulin peptides, proteolipid protein, myelin basic protein, villin, epithelial cellular adhesion molecule, collagen alpha-1, aggrecan core protein, 60 kDa chaperonin 2, vimentin, alpha-enolase, fibrinogen alpha chain, fibrinogen beta chain, chitinase-3-like protein, 60 kDa mitochondrial heat shock protein, matrix metalloproteinase-16, thyroid peroxidase, thyrotropin receptor, thyroglobulin, gluten, TSHR protein, glutamate decarboxylase 2, receptor-type tyrosine-protein phosphatase-like N, glucose-6-phosphatase 2, insulin isoform 2, zinc transporter 8, glutamate decarboxylase 1, GAD65, UniProt:A2RGMO, integrin alpha-Iib, integrin beta-3, EBV DNA polymerase catalytic subunit, 2′3′-cyclic-nucleotide 3′ phosphodiesterase, myelin associated oligodendrocyte basic protein, small nuclear ribonucleoprotein, U1 small nuclear ribonucleoprotein, histone H2B, histone H2A, histone H3.2, beta-2-glycoprotein, histone H4, 60S ribosomal protein L7, TNF-alpha, myeloperoxidase, Cbir1, MS4A12, DNA topoisomerase, CYP2D6, O-phosphoseryl-tRNA selenium transferase, pyruvate dehydrogenase complex, spectrin alpha chain, steroid 21-hydroxylase, acetylcholine receptor, MMP-16, keratin associated proteins. Chondroitin sulfate proteoglycan 4, myeloblastin, U1 small nuclear ribonucleoprotein 70 kDa, blood group Rh(D), blood group Rh(CE), myelin P2 protein, peripheral myelin protein 22, myelin protein P0, S-arrestin, collagen Alpha-1, coagulation factor VIII, collagen alpha-3(IV), desmoglein-3, desmoglein-1, Insulin-2, major DNA-binding protein, tyrosinase, 5,6-dihydroxyindole-2-carboxylic acid oxidase, HLA-A2, aquaporin-4, myelin proteolipid protein, ABC transporter, HLA I B-27 alpha chain, HLA I B-7 alpha chain, retinol-binding protein 3, or antigenic fragments thereof.

In some embodiments, the heterologous antigen is an antigen that is associated with an autoimmune disease. For example, in some embodiments, the heterologous antigen is associated with multiple sclerosis, diabetes mellitus Type I, rheumatoid arthritis, systemic lupus erythematosus, inflammatory bowel disease, celiac disease, Graves' disease, Hashimoto's autoimmune thyroiditis, vitiligo, rheumatic fever, pernicious anemia/atrophic gastritis, alopecia areata, immune thrombocytopenic purpura, temporal arteritis, ulcerative colitis, Crohn's disease, scleroderma, antiphospholipid syndrome, autoimmune hepatitis type 1, primary biliary cirrhosis, Sjogren's syndrome, Addison's disease, dermatitis herpetiformis, Kawasaki disease, sympathetic ophthalmia, HLA-B27 associated acute anterior uveitis, primary sclerosing cholangitis, discoid lupus erythematosus, polyarteritis nodosa, CREST Syndrome, myasthenia gravis, polymyositis/dermatomyositis, Still's disease, autoimmune hepatitis type 2, Wegener's granulomatosis, mixed Connective tissue disease, microscopic polyangiitis, autoimmune polyglandular syndrome, Felty's syndrome, autoimmune hemolytic anemia, chronic inflammatory demyelinating polyneuropathy, Guillain-Barre Syndrome, Behcet disease, autoimmune neutropenia, bullous pemphigoid, essential mixed cryoglobulinemia, linear morphea, autoimmune polyglandular syndrome 1 (APECED), acquired hemophilia A, Batten disease/neuronal ceroid lipofuscinoses, autoimmune pancreatitis, Hashimoto's encephalopathy, Goodpasture's disease, pemphigus vulgaris, autoimmune disseminated encephalomyelitis, relapsing polychondritis, Takayasu arteritis, Churg-Strauss syndrome, epidermolysis bullosa acquisita, cicatricial pemphigoid, pemphigus foliaceus, autoimmune hypoparathyroidism, autoimmune hypophysitis, autoimmune inner ear disease, autoimmune lymphoproliferative syndrome, autoimmune oophoritis, autoimmune orchitis, autoimmune polyglandular syndrome, Cogan's syndrome, encephalitis lethartica, erythema elevatum diutinum, Evans syndrome, immunodysregulation polyendocrinopathy enteropathy X-linked (IPEX), Issac's syndrome/acquired neuromyotonia, Miller Fisher syndrome, Morvan's syndrome, PANDAS, POEMS syndrome, Rasmussen's encephalitis, stiff-person syndrome, Vogt-Koyanagi-Harada syndrome, neuromyelitis optica, graft vs host disease, or autoimmune uveitis.

For example, in some embodiments the heterologous antigen is myelin oligodendrocyte glycoprotein, or an antigenic fragment thereof, which is associated with multiple sclerosis (MS). In some embodiments, the heterologous antigen is a pancreatic antigen, or antigenic fragment thereof, that is associated with Type I Diabetes (e.g., insulin)

In some embodiments, the heterologous antigen is an antigen, or antigenic fragment thereof, associated with a proliferative disorder such as cancer. For example, in some embodiments the heterologous antigen is associated with melanoma, basal cell carcinoma, squamous cell carcinoma, or testicular cancer. In some embodiments, the heterologous antigen is a melanocyte-specific antigen such as PMEL, TRP2, or MART-1. In some embodiments, the heterologous antigen is a testis cancer antigen such as NY-ESO or MAGE-A. In some embodiments, the heterologous antigen is a neoantigen. In some embodiments, the heterologous antigen is not a neoantigen.

In some embodiments, the heterologous antigen is a protein or antigenic peptide fragment thereof that is not natively expressed by either a commensal bacteria or a host. For example, in some embodiments, the heterologous antigen is gluten, or an antigenic fragment thereof, which is associated with celiac disease in a host.

In some embodiments, the heterologous antigen comprises a peptide having an amino acid sequence as listed in Table 2.

TABLE 2
EXEMPLARY HETEROLOGOUS ANTIGEN PEPTIDES AND AMINO
ACID SEQUENCES.

| Antigen | Amino Acid Sequence | SEQ ID NO. |
|---|---|---|
| OVA 323-329 | ISQAVHAAHAEINEAGR | 1 |
| MOG 35-55 | MEVGWYRSPFSRVVHLYRNGK | 2 |
| Insulin B9-23 (R22) | SHLVEALYLVCGEEG | 3 |
| epitope | | |
| ChgA epitope | SRLGLWVRME | 4 |
| 2.5HIP epitope | LQTLALWSRMD | 5 |
| PLP epitope 1 | ECCARCLVGAPFASLVATGLCFFG | 6 |
| PLP epitope 2 | LLLAEGFYTTGAVRQIFGDYK | 7 |
| PLP epitope 3 | VYIYFNTWTTCQSIAFPSKTSASIGSLCADAR | 8 |
| PLP epitope 4 | QMTFHLFIAAFVGAAATLVSLLTFM | 9 |
| MBP epitope | RPSQRSKYLATASTMDHARHG | 10 |
| Villin epitope 1 | KQHYLLYIWQGSQASQDEIAA | 11 |
| Villin epitope 2 | MSPKVDVFTANTSLSSGPLPTFPLEQL | 12 |
| Villin epitope 3 | STEDFTRALGMTPAAFSALPRWKQQ | 13 |
| Epcam epitope | VKGESLFHSSKSMDLRVNGE | 14 |

In some embodiments, the modified microorganism, e.g., a live, recombinant commensal bacteria, is capable of inducing a regulatory T cell response in the host to the heterologous antigen the modified microorganism is engineered to express. In other words, when the heterologous antigen is presented to a naïve T cell on the surface of an antigen presenting cell, the naïve T cell will differentiate into a T_{reg} cell. As is known in the art, differentiation into a T_{reg} cell can be induced under appropriate conditions, such as the presence of cytokines including TGF-β. Without intending to be bound by a particular mechanism, the modified microorganism, e.g. live, recombinant commensal bacteria, may induce production of cytokines by an APC that favor the differentiation of naïve T cells to T_{reg} cells. In some embodiments, the modified microorganism, e.g., a live, recombinant commensal bacteria, induces a T_{reg} response to the heterologous antigen, but does not elicit an immune response mediated by other subsets of T cells, such as CD8+ or Th17 T cells.

In some embodiments, the modified microorganisms, e.g., live, recombinant commensal bacteria, express the heterologous antigen at a level that is sufficient to trigger an immune response when the microorganism is engulfed by an antigen presenting cell (APC) and the antigen, or antigenic fragment thereof, is presented to a T cell in the context of an HLA molecule. Methods for optimizing protein expression levels in bacteria are described in Rosano G., et al. “Recombinant protein expression in Escherichia coli: advances and challenges,” Front Microbiol. 2014; 5: 172 (Published online 2014 Apr. 17).

In some embodiments, the heterologous antigen comprises non-natural amino acids. A “non-natural amino acid” refers to an amino acid that is not one of the 20 common amino acids and includes, but is not limited to, amino acids which occur naturally by modification of a naturally encoded amino acid (including but not limited to, the 20 common amino acids) but are not themselves incorporated into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine. Additionally, the term “non-natural amino acid” includes, but is not limited to, amino acids which do not occur naturally and may be obtained synthetically or may be obtained by modification of non-natural amino acids.

Expression of the heterologous antigen by the modified microorganisms, e.g., live, recombinant commensal bacteria, can be detected using assays that detect expression of the antigen RNA or protein, such as RT-PCR, Northern analysis, microarray, or Western blot.

In some embodiments, a heterologous antigen described herein is linked to an endogenous protein, or functional fragment of an endogenous protein, expressed by a commensal bacteria or bacterial strain. For example, in some embodiments, a heterologous protein, or antigenic fragment thereof, can be linked to an endogenous commensal bacterial protein, or functional fragment thereof, to form a fusion protein that is expressed by the live, recombinant commensal bacteria. In some embodiments, the heterologous protein, or antigenic fragment thereof, is fused to the N-terminus of the endogenous commensal bacterial protein, or functional fragment thereof. In some embodiments, the heterologous protein, or antigenic fragment thereof, is fused to the C-terminus of the endogenous commensal bacterial protein, or functional fragment thereof. In some embodiments, the heterologous antigen, or antigenic fragment thereof, can be linked to the endogenous commensal bacterial protein, or functional portion thereof, by an amino acid linker.

In some embodiments, the heterologous antigen, or antigenic fragment thereof, is linked to sialidase, endonuclease, secreted endoglycosidase, anti-sigma factor, thiol peroxidase, hypothetical protein BT_2621, hypothetical protein BT_3223, peptidase, Icc family phosphohydrolase, exo-poly-alpha-D-galacturonosidase, hypothetical protein BT_4428, or functional fragments thereof.

### 5. Nucleic Acids

In some embodiments, the modified microorganism, e.g., live, recombinant commensal bacteria, comprises a heterologous nucleic acid that is used to express a heterologous protein, or antigenic fragment thereof. In some embodiments, the heterologous nucleic acid is an RNA that is translated to produce a heterologous protein, or antigenic fragment thereof. In some embodiments, the heterologous nucleic acid is a DNA that encodes a heterologous protein, or antigenic fragment thereof (i.e., the DNA can be transcribed into mRNA that is translated to produce the heterologous protein or antigenic fragment thereof).

The heterologous nucleic acid typically includes regulatory sequences and coding region sequences. In some embodiments, the regulatory sequences are operably linked to the coding region sequences, such that the regulatory sequences control expression (e.g., transcription or translation) of the coding region sequences. The regulatory sequences can include sequence elements such as promoters and enhancers that bind regulatory proteins such as transcription factors and influence the rate of transcription of operably linked sequences. For example, the regulatory sequences can be located upstream (5′) or downstream (3′) of the coding region sequences, or both.

In some embodiments, the coding region sequences encode a heterologous protein that is useful for eliciting an immune response in a mammal. As is known by persons of skill in the art, various online servers can used to predict epitope-coding sequences that strongly bind to MHCII and elicit a T cell response (for example, see the Technical University of Denmark Department of Bio and Health Informatics NetMHCIIpan). The nucleic acid can also include sequences that, when transcribed and translated, provide signals for trafficking the heterologous protein to a specific cellular location or compartment (e.g., intracellular, secreted, or membrane bound).

In some embodiments, the heterologous nucleic acid is an expression vector comprising regulatory sequences that upregulate or downregulate transcription of the coding region sequence into RNA. In some embodiments, the modified microorganism, e.g., live recombinant commensal bacteria, comprises the necessary components to translate the RNA into protein, such as amino acids and tRNA. The expression vector can contain regulatory elements that direct expression of the heterologous antigen anywhere in the live, recombinant commensal bacterial, for example, the cytoplasm (soluble, not inclusion bodies), periplasm, fused to a cell surface protein, or secreted by the bacteria. Nucleic acid vectors for the expression of recombinant proteins in bacteria are well known by persons of skill in the art. For example, in some embodiments, the expression vector is pNBU2-bla-ermGb, pNBU2-bla-tetQb, or pExchange-tdk (see, for example, Wang J. et al. (2000). J Bacteriol. 182. 3559-71; pMM668, Addgene; Mimee M. et al. (2015) Cell Syst. 1(1):62-71; and Koropatkin N. et al. 2008. Structure. 16(7): 1105-1115).

In another example, in some embodiments, the expression vector is a pWW3837 vector (Genbank #KY776532), which is used to integrate an antigenic epitope coding region into the bacterial genome, as described in Whitaker et al., “Tunable Expression Tools Enable Single-Cell Strain Distinction in the Gut Microbiome,” Cell 169, 538-546, Apr. 20, 2017.

In some embodiments, the heterologous nucleic acid is stably integrated into the genome of the bacteria. In some embodiments, the heterologous nucleic acid is maintained as a plasmid in the bacteria. In some embodiments, the heterologous nucleic acid is an episomal plasmid.

In some embodiments, the heterologous nucleic acid comprises an epitope coding region sequence as listed in Table 3.

TABLE 3
REPRESENTATIVE HETEROLOGOUS EPITOPE CODING REGION
SEQUENCES.

| Antigen | Nucleotide Sequence | SEQ ID NO. |
|---|---|---|
| OVA 323-329 | ATTTCCCAGGCTGTTCATGCCGCACATGCTGA | 15 |
| | GATCAATGAGGCAGGACGT | |
| MOG 35-55 | ATGGAAGTCGGTTGGTATCGTTCCCCTTTTTCA | 16 |
| | CGTGTGGTGCATCTTTACCGCAACGGGAAA | |
| Insulin B9-23 (R22) | AGCCACTTAGTCGAAGCCCTTTACCTGGTTTG | 17 |
| epitope | CGGGGAAGAGGGT | |
| ChgA epitope | TCTCGCTTGGGATTATGGGTTCGTATGGAA | 18 |
| 2.5HIP epitope | TTGCAAACCTTGGCGCTGTGGTCGCGCATGGA | 19 |
| | T | |
| PLP epitope 1 | GAGTGTTGCGCCCGTTGCTTAGTTGGCGCTCC | 20 |
| | TTTCGCGTCATTGGTAGCCACGGGTTTGTGCTT | |
| | CTTTGGC | |
| PLP epitope 2 | TTACTTTTAGCGGAGGGCTTTTACACAACGGG | 21 |
| | TGCCGTTCGTCAGATCTTCGGTGACTATAAA | |
| PLP epitope 3 | GTATACATCTACTTCAACACGTGGACGACGTG | 22 |
| | TCAATCGATCGCCTTTCCGTCGAAGACTTCAG | |
| | CCTCTATTGGAAGCCTGTGCGCTGACGCCCGC | |
| PLP epitope 4 | CAGATGACCTTTCACCTGTTCATCGCGGCTTTT | 23 |
| | GTCGGAGCGGCTGCCACCTTAGTCAGTTTATT | |
| | AACATTTATG | |
| MBP epitope | CGCCCCAGCCAGCGCTCGAAATATCTGGCCAC | 24 |
| | AGCCTCAACAATGGATCATGCTCGCCACGGA | |
| Villin epitope 1 | AAACAGCACTACCTGTTATACATCTGGCAAGG | 25 |
| | CTCCCAGGCTTCTCAAGATGAAATTGCTGCT | |
| Villin epitope 2 | ATGAGTCCCAAAGTTGATGTTTTTACTGCAAA | 26 |
| | TACCTCCCTTAGTTCGGGACCTTTACCAACTTT | |
| | TCCCTTGGAACAGTTG | |
| Villin epitope 3 | TCCACTGAGGATTTCACACGCGCCCTTGGTAT | 27 |
| | GACCCCAGCAGCCTTCTCTGCTTTGCCACGTT | |
| | GGAAGCAACAG | |
| Epcam epitope | GTAAAAGGCGAATCCCTTTTCCACAGCTCTAA | 28 |
| | GTCGATGGATCTTCGTGTGAATGGAGAA | |

In some embodiments, the heterologous nucleic acid comprises non-natural nucleotides or analogues of natural nucleotides. Nucleotide analogs or non-natural nucleotides include nucleotides containing any type of modification to a base, sugar or phosphate moiety. Modifications can include chemical modifications. Modifications can be, for example, of the 3′OH or 5′OH groups of the backbone, sugar component or nucleotide base. Modifications may include the addition of non-naturally occurring linker molecules and/or cross-strand or intra-strand crosslinks. In one aspect, a modified nucleic acid comprises modification of one or more of a 3′OH or 5′OH group, backbone, sugar component, or nucleotide base, and/or addition of a non-naturally occurring linker molecule. In one aspect, the modified skeleton includes a skeleton other than the phosphodiester skeleton. In one aspect, modified sugars include sugars other than deoxyribose (in modified DNA) or sugars other than ribose (in modified RNA). In one aspect, modified bases include bases other than adenine, guanine, cytosine or thymine (in modified DNA) or bases other than adenine, guanine, cytosine or uracil (in modified RNA).

### 6. Methods of Producing Live, Recombinant Commensal Bacteria

Commensal bacteria can be engineered to express heterologous antigens, or antigenic fragments thereof, using general molecular biology methods as described in Green, M. R. and Sambrook, J., eds., Molecular Cloning: A Laboratory Manual, 4th ed., Cold Spring Harbor Laboratory Press, Cold Spring Harbor, N.Y. (2012), and Ausubel, F. M., et al. Current Protocols in Molecular Biology (Supplement 99), John Wiley & Sons, New York (2012), which are incorporated herein by reference.

To produce a live, recombinant, commensal bacterial strain that expresses a heterologous antigen or antigenic fragment thereof, antigenic epitope coding sequences can be cloned into an expression vector. A representative expression vector is the pWW3837 vector (Genbank #KY776532), (see Whitaker et al., “Tunable Expression Tools Enable Single-Cell Strain Distinction in the Gut Microbiome,” Cell 169, 538-546, Apr. 20, 2017). The antigenic epitope coding sequences can be cloned into the expression vector by known methods such as Gibson assembly. The expression vector can then be electroporated into a suitable bacterial donor strain, such as an Escherichia coli S17 lambda pir donor strain. The E. coli donor strain can be co-cultured overnight with recipient live commensal bacteria for conjugation, and positive colonies screened for incorporation of the expression vector.

Expression of the heterologous antigen can be determined by various assays, including detecting expression of the RNA encoding the antigen, for example, by Northern analysis or RT-PCR, or by detecting expression of the protein antigen, for example, by Western analysis.

### 7. Pharmaceutical Compositions

In some embodiments, provided in the present disclosure are pharmaceutical compositions comprising a modified microorganism, e.g. a live, recombinant commensal bacteria, as described herein and a pharmaceutically acceptable carrier. In some embodiments, the pharmaceutical composition induces an antigen-specific T cell response to a heterologous antigen expressed by the modified microorganism described herein when ingested by, or otherwise administered to, a subject. In some embodiments, the composition induces an antigen-specific T_{reg} response to the heterologous antigen expressed by the modified microorganism described herein. In some embodiments, the composition induces an antigen-specific T_{eff} response to the heterologous antigen expressed by the modified microorganism described herein.

In some embodiments, the pharmaceutical composition comprises a live, recombinant commensal bacteria comprising a heterologous nucleic acid that encodes a heterologous antigen that induces an antigen-specific T cell response when the composition is administered to a subject. In some embodiments, the pharmaceutical composition comprises a modified commensal bacteria comprising a heterologous nucleic acid that encodes a heterologous antigen that induces an antigen-specific T_{reg} response when the composition is administered to a subject. In some embodiments, the pharmaceutical composition comprises a modified commensal bacteria comprising a heterologous nucleic acid that encodes a heterologous antigen that induces an antigen-specific T_{eff} response when the composition is administered to a subject.

The pharmaceutical compositions described herein can include a pharmaceutically acceptable excipient. Examples of pharmaceutically acceptable excipients include, without limitation, sterile solutions such as water, saline, and phosphate buffered solutions. Additional examples of pharmaceutical excipients are described in the Handbook of Pharmaceutical Excipients, 8^{th} Edition, Authors/Editor: Sheskey, Paul J.; Cook, Walter G.; Cable, Colin G., Pharmaceutical Press (ISBN: 978-0-857-11271-2). It will be understood that the type of excipient used will depend on the route of administration to a subject.

In some embodiments, the pharmaceutical composition comprises a modified bacteria that is derived from a commensal bacteria that is native to the digestive tract of a mammal. In some embodiments, the pharmaceutical composition comprises a live, recombinant commensal bacterium selected from a Bacteroides sp. or Helicobacter sp. In some embodiments, the pharmaceutical composition comprises a recombinant B. thetaiotaomicron, B. vulgatus, B. finegoldii or H. hepaticus.

In some embodiments, the pharmaceutical composition comprises a modified bacteria that is derived from a commensal bacteria that is native to the skin of a mammal. In some embodiments, the pharmaceutical composition comprises a Staphylococcus spp. For example, in some embodiments, the pharmaceutical composition comprises a recombinant S. epidermidis.

The pharmaceutical composition disclosed herein can be administered to a subject via a suitable route that induces an antigen-specific immune response to the heterologous antigen, such as oral, nasal, subcutaneous, dermal, intradermal, intramuscular, mucosal or rectal.

In some embodiments, the pharmaceutical composition disclosed herein is administered to a subject via a suitable route to allow the modified microorganism, e.g., live, recombinant commensal bacterium, to colonize a niche in the subject that the microorganism from which the modified microorganism was derived would natively inhabit. For example, in some embodiments, the pharmaceutical composition disclosed herein is orally administered to a subject to allow a modified microorganism, e.g., a live recombinant bacterium derived from a commensal bacterium native to the gastrointestinal tract of the subject, to colonize the host's gastrointestinal tract. In some embodiments, for example, the pharmaceutical composition disclosed herein is topically administered to a subject to allow a modified microorganism, e.g., a live recombinant bacterium derived from a commensal bacterium native to the skin of the subject, to colonize the host's skin.

In some embodiments, the pharmaceutical composition comprises a material, such as a delayed-release enteric coating, that permits transit through the stomach to the small intestine before the modified microorganisms described herein, e.g. live, recombinant commensal bacteria, are released. Thus, in some embodiments, the pharmaceutical composition disclosed herein comprises an enteric-coated capsule containing a modified microorganism, e.g. a live, recombinant commensal bacterium, described herein. In some embodiments, the enteric coating comprises a polymer that is stable at an acidic pH, such as the acidic pH of the stomach, but breaks down or dissolves rapidly at an alkaline pH, such as the pH in the small intestine (pH 7-9).

In some embodiments, the pharmaceutical composition can further comprise additional agents that are useful for treating a disease or pathological condition in a subject. Examples of additional agents include small molecule drugs or antibodies that are useful for treating a disease or pathological condition in a subject.

### 8. Synthetic Bacterial Communities Comprising Bacteria that Induce a Regulatory T Cell Response

Modified microorganisms produced according to the disclosure (e.g., a live recombinant commensal bacteria) may be administered to a subject to induce an antigen-specific T cell immune response. It will be recognized that administering a cell does not generally refer to administration of a single cell, but encompasses administering a plurality of cells, typically a clonal population of cells with a desired property (i.e., expression of a heterologous antigen or antigenic fragment thereof).

U.S. Provisional Application No. 62/770,706, filed Nov. 21, 2018, and related International Patent Application No. PCT/US2019/062689, both entitled “High Complexity Synthetic Gut Bacterial Communities”, and the content of each of which is herein incorporated by reference in its entirety, describe defined stable microbial communities produced using in vitro and in vivo back-fill methods, i.e. “back-fill communities,” and methods for making such communities. These microbial communities comprise a cell of interest and are stable when engrafted into the mammalian (e.g., human) gut, such as a gut containing a human microbiome in the sense that the microbial ecosystem is at homeostasis such that a microbe of interest does not drop out of the community, is not over-grown by competing microbes in the gut, and does not overgrow and displace other microbes in the gut. If the combination of strains in the population is unstable, the population may change in unpredictable ways, which may change the metabolic phenotype of the community.

U.S. Provisional Application No. 62/770,706, and related International Patent Application No. PCT/US2019/062689, describe generation, screening and engraftment of communities with a desired “metabolic phenotype.” In one aspect, a metabolic phenotype may be the ability of a microbial strain or microbial community to transform one or more first compounds into one or more second compounds. For illustration, in one example a first compound(s) is enzymatically converted by the microbe or community into a second compound(s), and the metabolic phenotype is an increase in the amount of the second compound(s).

In some embodiments, a modified microorganism as described herein, e.g. a live, recombinant commensal bacteria, can be administered in combination with a high-complexity defined microbial community as disclosed in International Application No. PCT/US2019/062689. According to an aspect of the present disclosure, a desired phenotype of a high-complexity defined microbial community is the ability of a live, recombinant commensal bacterial cell as disclosed herein, to expresses a heterologous antigen, or antigenic fragment thereof, in sufficient amounts to induce an antigen-specific T cell response to the heterologous antigen. Thus, in one aspect of the present disclosure, a high-complexity defined microbial community comprising a modified microorganism, e.g., a live recombinant commensal bacteria, is administered to a subject (e.g., a mammal, such as a human) to allow colonization of a niche in the subject that a commensal bacteria from which the recombinant bacteria was derived would natively inhabit, resulting in induction of an antigen-specific T cell response to the heterologous antigen, or antigenic fragment thereof, expressed by the live recombinant commensal bacteria. In some embodiments, a high-complexity defined microbial community comprising a live, recombinant commensal bacteria described herein induces an antigen-specific regulatory T cell response in the subject into which the community is engrafted. In some embodiments, a high-complexity defined microbial community comprising a live, recombinant commensal bacteria described herein, induces an antigen-specific T effector cell response in the subject into which the community is engrafted.

One of ordinary skill in the art will appreciate that a high-complexity defined microbial community capable of inducing an antigen-specific T cell response to a heterologous antigen can produced as described in International Application No. PCT/US2019/062689, with the modification that the “metabolic phenotype” is the ability to elicit an antigen-specific T cell response. In this case, cultured or in vivo backfill communities are assayed for the ability to induce the desired antigen-specific T cell response. The desired antigen-specific T cell response may be considered a type of “metabolic phenotype.” Alternatively it is sometimes convenient to refer to the phenotype as an “immune phenotype.”

Assays for an immune phenotype are known in the art and are described in this disclosure including, without limitation, assays described in the section of this disclosure entitled “Methods for Detecting a T Cell Response.”

### 9. Methods of Inducing an Antigen-Specific T Cell Response

In another aspect, provided are methods for inducing an antigen-specific T cell response to a heterologous antigen, or antigenic fragment thereof, expressed by a modified microorganism, e.g. a live, recombinant commensal bacteria, as described herein. The methods can be performed in vitro or in vivo. In some embodiments, a live, recombinant commensal bacteria expressing a heterologous antigen of interest is contacted with an APC, wherein the APC phagocytizes the recombinant bacteria and processes the heterologous antigen, or antigenic fragment thereof, for presentation on MHC class I or MHC class II molecules. Examples of APCs include dendritic cells, macrophages, Langerhans cells, B cells, intestinal epithelial cells, and innate lymphoid cells. In some embodiments, the APC is a dendritic cell, such as a CD103+CD11b+ dendritic cell. In some embodiments, the APC is an intestinal macrophage, such as a CX3CR1+ intestinal macrophage.

In some embodiments, the APC displaying the processed heterologous antigen in complex with an MHC molecule on its cell surface is then contacted with a T cell, such as a naïve T cell. In some embodiments, binding of the processed heterologous antigen/MHC complex to the T Cell Receptor (TCR) on the naïve T cell results in differentiation of the naïve T cell into a regulatory T cell (Treg). In some embodiments, activation of the T Cell Receptor (TCR) of the naïve T cell results in differentiation of the naïve T cell into a regulatory T cell (T_{reg}). In some embodiments, binding of the processed heterologous antigen/MHC complex to the T Cell Receptor (TCR) on the naïve T cell results in differentiation of the naïve T cell into an effector T cell (T_{eff}).

The induction of an antigen-specific T cell response can be detected using a suitable assay, such as cell surface marker expression analysis (e.g., by flow cytometry analysis) for specific T cell sub-populations. Suitable assays for detecting T_{reg} cells are described herein.

In an in vitro method of inducing an antigen-specific T Cell response, live, recombinant commensal bacteria expressing a heterologous antigen of interest are cultured with APCs in a suitable media under conditions that permit the APC to phagocytize the bacteria, process the heterologous antigen, and display the processed antigen on the cell surface. Naïve T cells can be added to the in vitro culture of APCs and bacteria, or the APCs can be isolated from the bacteria and cultured with the naïve T cells. The media can contain growth factors and cytokines that promote survival and differentiation of the T cells into a given T cell subset. In some embodiments, the media contains factors that promote the differentiation of T_{reg} cells, such as TGF-β. In some embodiments, the media contains factors that promote the differentiation of T_{eff} cells, such as IL-12, IL-2, and IFNγ.

In some embodiments, the T cells are primary T cells. In some embodiments, the T cells are primary T cells isolated from the gut or spleen of a subject. In some embodiments, the isolated T cells include fully differentiated T_{regs}. In some embodiments, freshly isolated primary T cells are cultured in basic medium (i.e. DMEM+5% FBS) without growth factors or cytokines.

In another embodiment of inducing an antigen-specific T cell response, the method is an in vivo method. In some embodiments, a subject or patient is administered a pharmaceutical composition comprising a modified microorganism, e.g., a live, recombinant commensal bacteria expressing a heterologous antigen of interest. The pharmaceutical composition can be administered by any suitable route, further described herein. For example, in some embodiments the pharmaceutical composition is ingested by the subject for delivery of the recombinant bacteria to a native gastrointestinal niche in the subject. In some embodiments, for example, the pharmaceutical composition is administered topically for delivery of the recombinant bacteria to an epidermal niche on the subject. While not being bound by theory, it is expected that after the pharmaceutical composition is administered to the subject, the modified microorganism, e.g., the live recombinant commensal bacteria, expressing the heterologous antigen of interest will be phagocytized by an APC in the subject, processed, and presented to naïve T-cells in the subject, thereby inducing an antigen-specific T cell response. In some embodiments, administration of the pharmaceutical composition elicits an antigen-specific T_{reg} response. In some embodiments, administration of the pharmaceutical composition elicits a T_{eff} response.

In some embodiments, differentiation into T_{regs} is influenced by the type of bacteria engulfed by an APC. In some embodiments, a heterologous antigen can induce the differentiation of different T cell populations depending on the bacterial strain the heterologous antigen is expressed in. For example, in some embodiments, a live, recombinant commensal bacteria derived from a bacterial strain that is commensal to a mammalian gut niche can induce a Treg response specific for the heterologous antigen expressed by the recombinant bacteria, whereas the same heterologous antigen when expressed in a live, recombinant commensal bacteria derived from a bacterial strain that is commensal to a skin niche of a mammal induces the generation of an antigen-specific CD8+T_{eff} response.

### 10. Methods for Detecting a T Cell Response

An antigen-specific T cell response to the heterologous antigen can be detected by a variety of techniques known in the art. For example, the T cell response can be detected by isolating lymphocytes from a subject administered with a live, recombinant commensal bacteria disclosed herein, or a pharmaceutical composition comprising the same, and assaying the lymphocytes ex vivo for the presence of antigen-specific T cells. Methods for detecting antigen-specific T cells isolated from human subjects are described, for example, in the “Manual of Molecular and Clinical Laboratory Immunology, 7^{th} Edition,” Editors: B. Detrick, R. G. Hamilton, and J. D. Folds, 2006, e-ISBN: 9781555815905.

Methods for detecting a T cell response to antigens include flow cytometry, cytokine assays (e.g. ELISA) and TCR sequencing. Flow cytometry can be used to detect expression of cell surface and/or intracellular markers before and after differentiation of a naïve T cell into an activated T cell. For example, to detect an antigen-specific T_{reg} response, the cells can be labeled with antibodies that bind CD3, CD4, CD25, FOXP3, and CD127, and gated on cells that are CD3+, CD4+, CD25hi, FOXP3+, and CD127lo. Because activated T cells often up-regulate CD25, and Foxp3 is expressed by effector (non-suppressive) T cell lineages, another gating strategy is to omit Foxp3 and sort cells that are CD3+, CD4+, CD25hi, and CD12710 cells. The population of sorted cells can then be assayed for T_{reg} properties, for example, by cytokine analysis and/or suppression co-culture assays with non-T_{reg} T cells (CD3+CD4+CD25-, CD127hi). Inducible T_{regs} can also be detected by analyzing for expression of both RORγt and Foxp3 (see Xu M. et al., “c-Maf-dependent regulatory T cells mediate immunological tolerance to a gut pathobiont,” Nature. 2018 Feb. 15; 554(7692): 373-377).

Other assays to detect antigen-specific T_{reg} cells include suppression assays. For example, responder CD4+ T cells are stimulated polyclonally and cocultured with different ratios of putative T_{reg} cells, and the cultures are treated with ³H-thymidine to monitor DNA synthesis of responder T cells. T_{reg} cells can also be detected by measuring the production of cytokines IL-2 and IFN-γ in the coculture assays, as the level of these cytokines is decreased by T_{reg} suppression of responder T cells. Another assay to detect an antigen-specific T_{reg} response is to detect the expression of IL-2 and IFN-γ mRNA or CD69 and CD154 surface protein expression in responder T cells, where suppression can be detected within 5-7 hours of coculturing the responder T cells with putative T_{reg} cells. (See McMurchy et al., “Suppression assays with human T regulatory cells: A technical guide,” Eur. J. Immunol. 2012. 42: 27-34), which is incorporated by reference herein.

Additional assays to detect an antigen-specific T_{reg} responses include sequence analysis of single cell mRNA as described in Miragaia et al., “Single-Cell Transcriptomics of Regulatory T Cells Reveals Trajectories of Tissue Adaptation,” Immunity 50, 493-504, Feb. 19, 2019; and transcriptome profiling as described in Bhairavabhotla et al., Transcriptome Profiling of Human FoxP3+ Regulatory T Cells,” Human Immunology, Volume 77, Issue 2, February 2016, Pages 201-213. Another assay for detecting an antigen-specific T_{reg} response comprises sequencing the TCR of T_{reg} cells, as described in Rossetti et al., “TCR repertoire sequencing identifies synovial T_{reg} cell clonotypes in the bloodstream during active inflammation in human arthritis,” Ann Rheum Dis 2017; 76:435-441 (doi:10.1136/annrheumdis-2015-208992).

Yet another assay for detecting an antigen-specific T_{reg} response involves detecting DNA methylation of the FoxP3 locus in T cells, as described in Baron U. et al., “DNA demethylation in the human FOXP3 locus discriminates regulatory T cells from activated FOXP3(+) conventional T cells,” Eur J Immunol 2007; 37:2378-89 (doi:10.1002/eji.200737594). In some embodiments, the assay for detecting an antigen-specific T_{reg} response uses an APC, heterologous antigen (or heterologous antigen expressing bacteria) and T cell co-culture system. After a suitable period of co-culture (e.g., about 1, 2, 3, 4, or 5 hours of co-culture), expression of Nur77 is monitored to detect antigen-specific TCR activation.

To detect an antigen-specific T_{eff} response, cells can be labeled with antibodies that bind to T cell markers that are characteristic of specific T cell lineages and the proportion of different T cell subset populations can be analyzed using techniques known by persons of skill in the art (e.g., see Syrbe, et al. (1999) Springer Semin Immunopathol 21, 263-285; Luckheeram R V et al. (2012). Clin Dev Immunol. 2012; 2012:925135; Mahnke Y D et al. (2013) Cytometry A 83(5):439-440). For example, in some embodiments, cells can be labelled with one or more antibodies that bind CD3, CD8, CCR7, IFNγ, T-bet, CXCR3, CCR5, IL-4, IL-5, GATA3, STAT6, CCR4, CCR8, IL-17, RORγT, or CCR6. In a further example, to identify CD8+ T cells, cells can be labeled with antibodies that bind CD3, CD8, and CCR7 and gated on cells that are CD3+, CD8+, and CCR7−.

Assays for detecting an antigen-specific T_{eff} response are well known by persons of skill in the art. For example, in some embodiments, the assay for detecting an antigen-specific T_{eff} response uses an APC, heterologous antigen (or heterologous antigen expressing bacteria) and T cell co-culture system. After a suitable period of co-culture (e.g., about 1, 2, 3, 4, or 5 hours of co-culture), expression of Nur77 is monitored to detect antigen-specific TCR activation (e.g., see Ashouri J F and Weiss A (2017) J Immunol. 198 (2) 657-668).

Other assays to detect antigen-specific T_{eff} cells include proliferation assays. For example, responder CD8+ T cells are stimulated polyclonally and cocultured with different ratios of putative T_{eff} cells, and the cultures are treated with ³H-thymidine to monitor DNA synthesis of responder T cells. T_{eff} cells can also be detected by measuring the production of cytokines (e.g., IFN-γ) in coculture assays, as well as measuring the production of perforin and granzyme.

### 11. Methods of Treatment

Also provided are methods of preventing or treating a disease, disorder or condition in a subject or patient with a pharmaceutical composition described herein. In some embodiments, the method comprises administering a therapeutically effective amount of a pharmaceutical composition comprising a modified microorganism, e.g., a live recombinant commensal bacterial cell or strain, described herein to the subject. The pharmaceutical composition can be administered to the subject by any suitable route that does not trigger an adverse reaction in the subject. For example, the pharmaceutical composition can be administered by oral, nasal, vaginal, rectal, topical, subcutaneous, intradermal or intramuscular routes. In some embodiments, the pharmaceutical composition is ingested orally by the subject, administered topically to the subject, inhaled by the subject, or injected into the subject. In some embodiments, the pharmaceutical composition is administered in a material, such as a delayed release enteric coating, that permits transit through the stomach to the small intestine before the pharmaceutical is released. Thus, in some embodiments, the pharmaceutical composition comprises a enteric-coated capsule containing a modified microorganism, e.g., a live, recombinant commensal bacteria described herein.

In some embodiments, pharmaceutical compositions comprising a modified microorganism, e.g., a live recombinant commensal bacteria, described herein, is used for the prevention or treatment of an autoimmune disease. Examples of autoimmune diseases that can be treated by a modified microorganism disclosed herein include multiple sclerosis, diabetes mellitus Type I, rheumatoid arthritis, systemic lupus erythematosus, inflammatory bowel disease, celiac disease, Graves' disease, Hashimoto's autoimmune thyroiditis, vitiligo, rheumatic fever, pernicious anemia/atrophic gastritis, alopecia areata, immune thrombocytopenic purpura, temporal arteritis, ulcerative colitis, Crohn's disease, scleroderma, antiphospholipid syndrome, autoimmune hepatitis type 1, primary biliary cirrhosis, Sjogren's syndrome, Addison's disease, dermatitis herpetiformis, Kawasaki disease, sympathetic ophthalmia, HLA-B27 associated acute anterior uveitis, primary sclerosing cholangitis, discoid lupus erythematosus, polyarteritis nodosa, CREST Syndrome, myasthenia gravis, polymyositis/dermatomyositis, Still's disease, autoimmune hepatitis type 2, Wegener's granulomatosis, mixed Connective tissue disease, microscopic polyangiitis, autoimmune polyglandular syndrome, Felty's syndrome, autoimmune hemolytic anemia, chronic inflammatory demyelinating polyneuropathy, Guillain-Barre Syndrome, Behcet disease, autoimmune neutropenia, bullous pemphigoid, essential mixed cryoglobulinemia, linear morphea, autoimmune polyglandular syndrome 1 (APECED), acquired hemophilia A, Batten disease/neuronal ceroid lipofuscinoses, autoimmune pancreatitis, Hashimoto's encephalopathy, Goodpasture's disease, pemphigus vulgaris, autoimmune disseminated encephalomyelitis, relapsing polychondritis, Takayasu arteritis, Churg-Strauss syndrome, epidermolysis bullosa acquisita, cicatricial pemphigoid, pemphigus foliaceus, autoimmune hypoparathyroidism, autoimmune hypophysitis, autoimmune inner ear disease, autoimmune lymphoproliferative syndrome, autoimmune oophoritis, autoimmune orchitis, autoimmune polyglandular syndrome, Cogan's syndrome, encephalitis lethartica, erythema elevatum diutinum, Evans syndrome, immunodysregulation polyendocrinopathy enteropathy X-linked (IPEX), Issac's syndrome/acquired neuromyotonia, Miller Fisher syndrome, Morvan's syndrome, PANDAS, POEMS syndrome, Rasmussen's encephalitis, stiff-person syndrome, Vogt-Koyanagi-Harada syndrome, neuromyelitis optica, graft vs host disease, and autoimmune uveitis.

In some embodiments, pharmaceutical compositions comprising a modified microorganism, e.g., a live recombinant commensal bacteria, described herein, is used for the prevention or treatment of a proliferative disease. Examples of proliferative diseases include melanoma, basal cell carcinoma, squamous cell carcinoma, and testicular cancer.

Any suitable animal model can be used to test the methods described herein. In some embodiments, the animal model is a mouse model, or a non-human primate model.

### 12. Kits Comprising the Bacterial Strains

In another aspect, a kit comprising the modified microorganism, e.g., the live recombinant commensal bacteria is provided. The kit can include a live, recombinant commensal bacterial that expresses a heterologous antigen described herein. In some embodiments, the heterologous antigen is an antigen normally present in a non-bacterial host of the commensal bacteria. For example, the heterologous antigen can be an antigen that is expressed by or present in a vertebrate or mammal.

In some embodiments, a kit comprises a pharmaceutical composition described herein. For example, the kit can include a pharmaceutical composition comprising a modified microogranisma, e.g., a live, recombinant commensal bacteria that expresses a heterologous antigen. In some embodiments, the pharmaceutical composition is capable of inducing a regulatory T cell response to the heterologous antigen. In some embodiments, the pharmaceutical composition is capable of inducing an effector T cell response of the heterologous antigen.

In some embodiments, the kit can also include instructions for administering the pharmaceutical composition to a subject or patient. In addition, the kit can include pharmaceutical excipients that aid in administering the pharmaceutical compositions.

In some embodiments, the kit can also include additional agents that are useful for treating a disease or pathological condition in a subject. Examples of additional agents include small molecule drugs or antibodies that are useful for treating a disease or pathological condition in a subject.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1—Expression of OVA in Bacteroides Strains

Antigenic epitope coding sequences were cloned into the pWW3837 vector (Genbank #KY776532), (see Whitaker et al., “Tunable Expression Tools Enable Single-Cell Strain Distinction in the Gut Microbiome,” Cell 169, 538-546, Apr. 20, 2017) by Gibson assembly. The vector was electroporated into E. coli S17 lambda pir donor strains. E. coli donor strains were co-cultured overnight with recipient bacteria for conjugation on a BHI blood plate. Biomass was scraped and plated onto BHI Blood+erm/gent plates. Positive colonies were screened by colony-PCR.

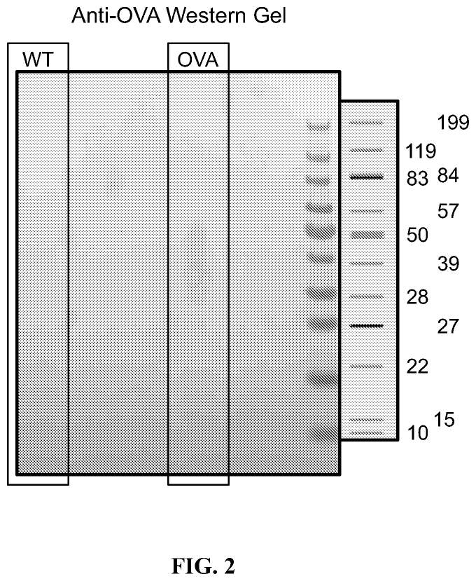

As shown in FIG. 2, Western blotting data demonstrates that Bacteroides thetaiotaomicron engineered to express an OVA epitope (OVA+B. thetaiotaomicron) showed detectable levels of OVA whereas wild-type B. thetaiotaomicron (WT B. thetaiotaomicron; negative control) shows no signal.

### Example 2—In Vitro Induction of OVA-Specific T Cells by Recombinant Bacteroides Strains

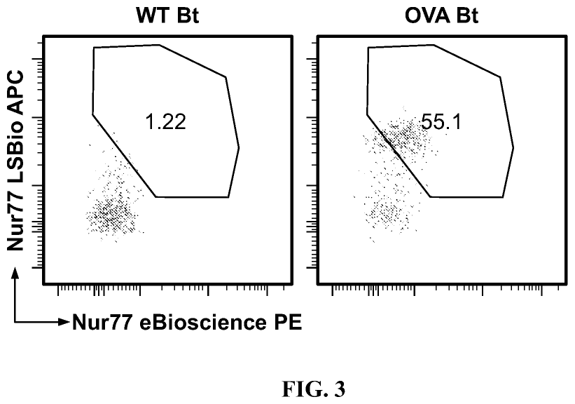

OVA-specific T cells isolated from the spleens of OTII transgenic mice were co-cultured for 4 hours with B16-FLT3L stimulated DCs and OVA+B. thetaiotaomicron or WT B. thetaiotaomicron. As shown in FIG. 3, OTII T cells cultured with OVA+B. thetaiotaomicron upregulate the expression of Nur77 (two different Nur77 antibodies were used to increase specificity).

### Example 3—Expression of MOG Fusion Peptides in Recombinant Bacteroides Strains

Myelin oligodendrocyte glycoprotein (MOG) 35-55 peptide sequences were cloned into the pWW3837 vector, electroporated into E. coli donor strains, and conjugated with commensal recipient strains using an analogous method as described in Example 1.

Commensal bacterial strains and expression constructs are summarized in Table 4.

TABLE 4
MOG-Expressing Bacterial Strains and Constructs

| | | | Location of MOG |
|---|---|---|---|
| | | | Peptide Relative to |
| | | Native Fusion | Native Fusion |
| Strain Name | Commensal Strain | Protein | Protein |
| BT_W | Bacteroides thetaiotaomicron | — | — |
| | VPI-5482 | | |
| BT_MOG#1 | Bacteroides thetaiotaomicron | BT0455 (Sialidase) | N-Terminal |
| | VPI-5482 | | |
| BT_M0G#5 | Bacteroides thetaiotaomicron | BT1279 (Anti-Sigma | N-Terminal |
| | VPI-5482 | Factor) | |
| BV_W | Bacteroides vulgatus | — | — |
| | ATCC 8482 | | |
| BV_MOG#1 | Bacteroides vulgatus | BT0455 (Sialidase) | N-Terminal |
| | ATCC 8482 | | |
| BV_MOG#5 | Bacteroides vulgatus | BT1279 (Anti-Sigma | N-Terminal |
| | ATCC 8482 | Factor) | |
| BF_W | Bacteroides finegoldii | — | — |
| | DSM 17565 | | |
| BF_MOG#1 | Bacteroides finegoldii | BT0455 (Sialidase) | N-Terminal |
| | DSM 17565 | | |
| BF_M0G#5 | Bacteroides finegoldii | BT1279 (Anti-Sigma | N-Terminal |
| | DSM 17565 | Factor) | |

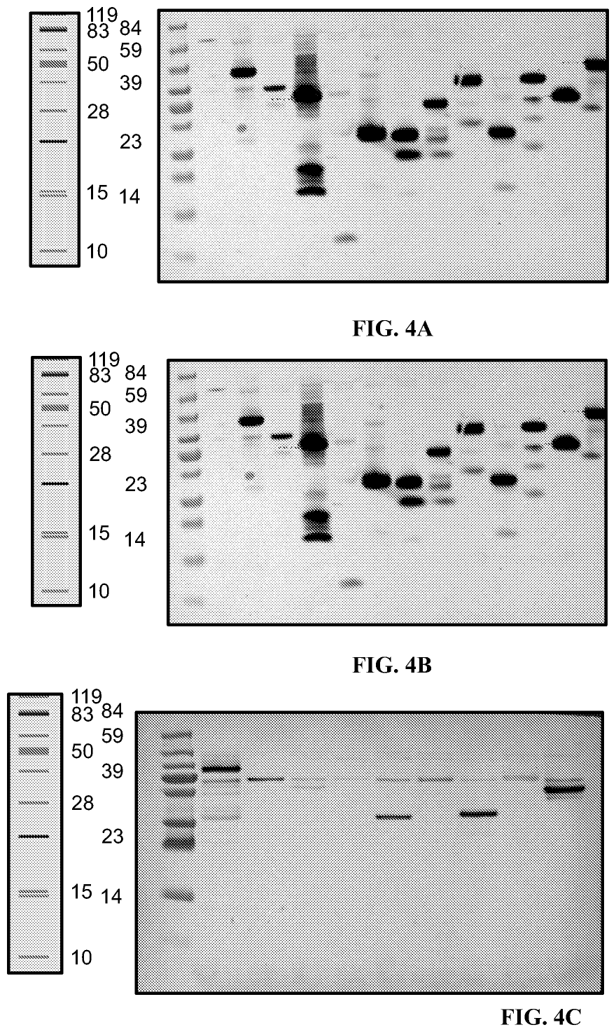

As shown in FIG. 4, Western blotting data using an anti-FLAG antibody demonstrates that B. thetaiotaomicron (FIG. 4A) engineered to express FLAG-tagged MOG35-55 peptide (BT_MOG #1 and BT_MOG #5), Bacteroides vulgatus (FIG. 4B) engineered to express FLAG-tagged MOG 35-55 peptide (BV_MOG #1 and BT_MOG #5), and Bacteroides finegoldii (FIG. 4C) engineered to express FLAG-tagged MOG 35-55 peptide (BF_MOG #1 and BF_MOG #5), all showed detectable levels of MOG peptide whereas wild-type B. thetaiotaomicron, B. vulgatus, and B. finegoldii (BT_W, BV_W, and BF_W, respectively), did not show any signal.

### Example 4—In Vitro Induction of MOG-Specific T Cells by Recombinant Bacteroides Strains

To expand splenic dendritic cells (DCs), CD45.1 C57BL/6 (The Jackson Laboratory, strain #002014) mice were injected subcutaneously at the flank with 5×10⁶ B16 melanoma cells overexpressing Flt3L. On day 11, spleens were harvested, digested using a spleen dissociation kit (Miltenyi) and splenic DCs were purified using CD11c microbeads (Miltenyi).

To prepare bacterial antigen, live, recombinant B. thetaiotaomicron expressing MOG35-55 peptide (prepared by a method analogous to the method described in Example 3) were washed and resuspended in complete T cell media (DMEM, 10% FBS, 10 mM HEPES, 50 μM 2-ME). Heat-killing was performed at 65° C. for 15 minutes and loss of bacterial viability was confirmed by culturing. Autoclaved antigen was prepared by autoclaving bacterial suspension at 121° C. for 45 minutes at 15 psi. MOG-specific T cells were isolated and purified from spleens and peripheral lymph nodes of 2D2 TCR-Tg mice (The Jackson Laboratory, strain #006912) using a CD4 T cell isolation kit (Miltenyi).

To prepare APC-T cell co-cultures, 2×10⁵ splenic DCs were pulsed with live, heat-killed or autoclaved bacteria at a multiplicity of infection (MOI) of 10-50 or 40 μg/ml of total protein for 4 hours at 37° C. 2×10⁵ MOG-specific 2D2 CD4 T cells were added to APCs. On day 2 post-co-culture, cells were harvested, stained with fluorochrome conjugated antibodies for CD45.1, CD45.2, TCRb, CD4, CD25, CD44, CD69 (ThermoFisher Scientific or BioLegend) and assessed by flow cytometry (Attune NxT). Live cells were excluded by Live/Dead Aqua (ThermoFisher Scientific). Data analysis was performed using FlowJo v10.

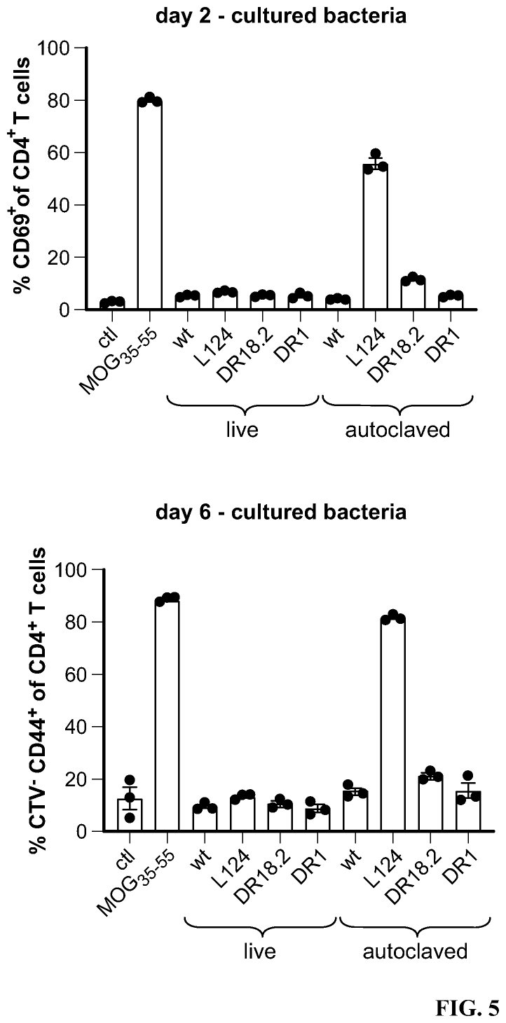

As shown in FIGS. 5A and 5B, recombinant B. thetaiotaomicron strains expressing MOG35-55 peptide (L124, DR18.2, and DR1) induced a greater antigen-specific induction of CD4+ T cells than wild-type B. thetaiotaomicron (wt).

### Example 5—In Vivo Induction of MOG-Specific T Cells by Recombinant Bacteroides Strains

The Experimental Autoimmune Encephalomyelitis (EAE) model was used as a murine model for multiple sclerosis (MS). Germ-free 8-10 week old C57BL/6 mice or C57BL/6-Tg (Tcra2D2,Tcrb2D2)1Kuch/J mice were orally inoculated with MOG35-55 peptide-expressing bacteria (BVF-MOG=a mixture of B. vulgatus and B. finegoldii expressing MOG35-55) or wild-type commensal bacteria as a negative control (BVF-WT=a mixture of wild-type B. vulgatus and B. finegoldii) on day one. Wild-type and recombinant bacterial strains were obtained as previously described in Example 3. On day 14, these mice were subcutaneously immunized with the Hooke Kit™MOG35-55/CFA emulsion (EK-2110, Hooke Labs, St Lawrence, Mass., USA), which contains 200 μg MOG35-55 emulsified in 2004 Complete Freund's Adjuvant (CFA). On day 14, 2 hours after MOG35-55/CFA immunization, 200 ng of pertussis toxin (PTX) in phosphate buffered saline (PBS) was injected into the intraperitoneal cavity of each mouse. On day 15, 200 ng of pertussis toxin (PTX) in PBS was injected intraperitoneally. EAE scores and body weights were assessed daily from day 15 to day 34 in order to evaluate the severity and stage of the disease. To alleviate the distress from this experiment, mice were euthanized when reaching a score of 3.5. Score 0 means no obvious changes in motor functions. Score 0.5 is a distal paralysis of the tail; score 1 complete tail paralysis; score 1.5 mild paresis of one or both hind legs; score 2 severe paresis of hind legs; score 2.5 complete paralysis of one hindleg; score 3 complete paralysis of both hind legs and score 3.5 complete paralysis of hind legs and paresis of one front leg. Mice reaching scores ≥3.5 will be euthanized.

On day 35, mice were euthanized; spinal cord samples were prepared for histological analysis; inguinal lymph nodes were collected, washed with PBS, dissociated to obtain a cell suspension, fixed used a FoxP3 staining buffer set (eBioscience), and stained with various fluorescently-labelled antibodies for flow cytometry analysis on a BD-LSRII instrument.

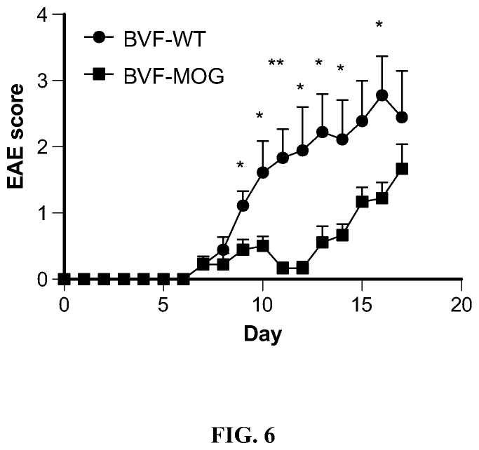

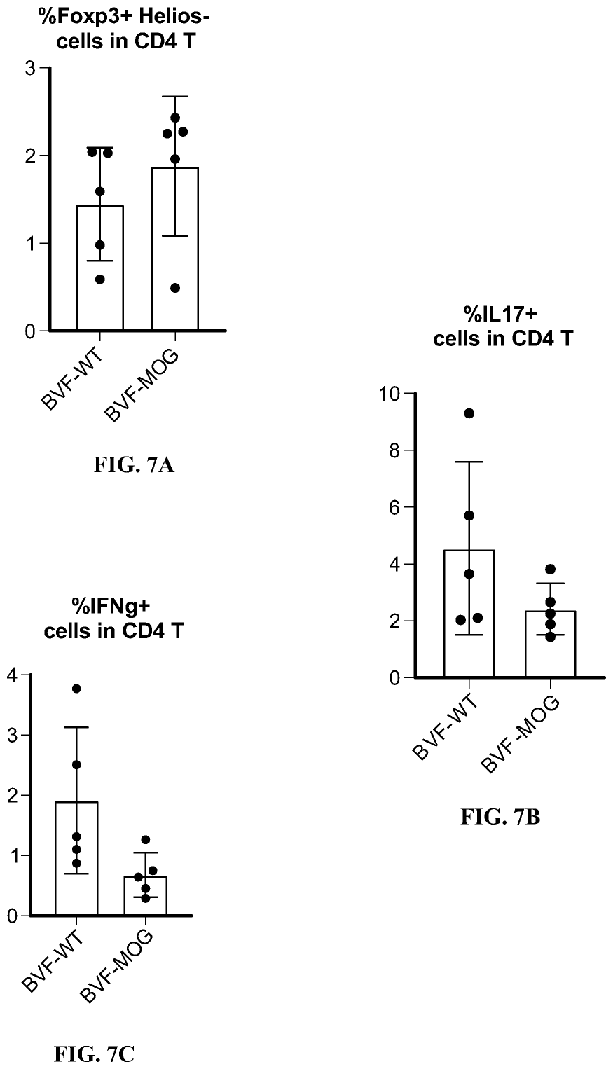

As shown in FIG. 6, mice administered with a mixture of recombinant B. vulgatus and B. finegoldii expressing MOG35-55 peptide (BVF-MOG) had a significantly reduced EAE score as compared to mice administered with a mixture of wild-type B. vulgatus and B. finegoldii (BVF-WT). *p≤0.05, **p≤0.01. Results are from three independent experiments. As shown in FIG. 7A, mice administered with a mixture of recombinant B. vulgatus and B. finegoldii expressing MOG35-55 peptide (BVF-MOG) had an increased number of lymph node FoxP3+Helios-CD4+ T cells as compared to mice administered with a mixture of wild-type B. vulgatus and B. finegoldii (BVF-WT). Mice administered with a mixture of recombinant B. vulgatus and B. finegoldii expressing MOG35-55 peptide (BVF-MOG) also exhibited fewer IL17+CD4+ T cells (FIG. 7B) and IFN-γ+CD4+ T cells (FIG. 7C) as compared to mice administered with a mixture of wild-type B. vulgatus and B. finegoldii (BVF-WT).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A live, recombinant commensal bacterium, wherein the bacterium is engineered to express a non-native protein or peptide, wherein the protein or peptide is associated with a host disease or condition, wherein upon administration of the bacterium to the host resulting in colonization of a native host niche by the bacterium, the host mounts an adaptive immune response to the non-native protein or peptide, wherein the adaptive immune response is a regulatory T-cell (Treg) response or an effector T-cell (Teffector) response.

2. The recombinant commensal bacterium of claim 1, wherein the colonization of the native host niche is persistent or transient.

3. The recombinant commensal bacterium of claim 2, wherein the native host niche is transiently colonized, and wherein colonization is for 1 day to 60 days.

4. The recombinant commensal bacterium of claim 2, wherein the native host niche is transiently colonized, and wherein colonization is for 3.5 days to 60 days.

5. The recombinant commensal bacterium of claim 3 or 4, wherein the native host niche is transiently colonized, and wherein colonization is for 7 days to 28 days.

6. The recombinant commensal bacterium of any one of claims 1-5, wherein colonization is determined by polymerase chain reaction or colony forming assay performed on a sample obtained from the host after 1 day, 3.5 days, 7 days, 14 days, 28 days, or 60 days after administration to the host.

7. The recombinant commensal bacterium of any one of claims 1-6, wherein the administration results in interaction of the bacterium with a native immune system partner cell.

8. The recombinant commensal bacterium of claim 7, wherein the native immune system partner cell is an antigen-presenting cell.

9. The recombinant commensal bacterium of claim 8, wherein the antigen-presenting cell is selected from the group consisting of a dendritic cell, a macrophage, a B-cell, and an intestinal epithelial cell.

10. The recombinant commensal bacterium of any one of claims 1-9, wherein the native host niche is selected from the group consisting of the gastrointestinal tract, respiratory tract, urogenital tract, and skin.

11. The recombinant commensal bacterium of any one of claims 1-10, wherein the non-native protein or peptide is a host protein or peptide.

12. The recombinant commensal bacterium of any one of claims 1-11, wherein the bacterium is a Gram-negative bacterium.

13. The recombinant commensal bacterium of claim 12, wherein the Gram-negative bacterium is selected from the group consisting of Bacteroides thetaiotaomicron, Helicobacter hepaticus and Parabacteroides sp.

14. The recombinant commensal bacterium of any one of claims 1-11, wherein the bacterium is a Gram-positive bacterium.

15. The recombinant commensal bacterium of claim 14, wherein the Gram-positive bacterium is selected from the group consisting of Staphylococcus epidermidis, Faecalibacterium sp. and Clostridium sp.

16. The recombinant commensal bacterium of any one of claims 1-11, wherein the administration is via a route selected from the group consisting of topical, enteral, parenteral and inhalation.

17. The recombinant commensal bacterium of claim 16, wherein the route is topical.

18. The recombinant commensal bacterium of claim 17, wherein the bacterium is S. epidermidis.

19. The recombinant commensal bacterium of claim 16, wherein the route is enteral.

20. The recombinant commensal bacterium of claim 19, wherein the bacterium is selected from the group consisting of Bacteroides spp., Clostridium spp., Helicobacter spp., Parabacteroides spp, and Prevotella spp.

21. The recombinant commensal bacterium of claim 20, wherein the bacterium is selected from the group consisting of Bacteroides thetaiotaomicron, Bacteroides vulgatus and Bacteroides finegoldii.

22. The recombinant commensal bacterium of any one of claims 1-11, wherein the adaptive immune response is a Treg response and the bacterium is selected from the group consisting of Bacteroides spp., Helicobacter spp., Parabacteroides spp., Clostridium spp., Staphylococcus spp., Lactobacillus spp., Fusobacterium spp., Enterococcus spp., Acenitobacter spp., Flavinofractor spp., Lachnospiraceae spp., Erysipelotrichaceae spp., Anaerostipes spp., Anaerotruncus spp., Coprococcus spp., Clostridiales spp., Odoribacter spp., Collinsella spp., Bifidobacterium spp., Streptococcus spp., and Prevotella spp.

23. The recombinant commensal bacterium of claim 22, wherein the adaptive immune response is a Treg response and the bacterium is selected from the group consisting of Clostridium ramosum, Staphylococcus saprophyticus, Bacteroides thetaiotaomicron, Clostridium histolyticum, Lactobacillus rhamnosus, Parabacteroides johnsonii, Fusobacterium nucleatum, Enterococcus faecium, Lactobacillus casei, Acenitobacter lwofii, Bacteroides ovatus, Bacteroides vulgatus, Bacteroides uniformis, Bacteroides finegoldii, Clostridium spiroforme, Flavonifractor plautii, Clostridium hathewayi, Lachnospiraceae bacterium, Clostridium bolteae, Erysipelotrichaceae bacterium, Anaerostipes caccae, Anaerotruncus colihominis, Coprococcus comes, Clostridium asparagiforme, Clostridium symbiosum, Clostridium ramosum, Clostridium sp. D5, Clostridium scindens, Lachnospiraceae bacterium, Clostridiales bacterium, Bacteroides intestinalis, Bacteroides caccae, Bacteroides massiliensis, Parabacteroides distasonis, Odoribacter splanchnicus, Collinsella aerofaciens, Acinetobacter lwoffii, Bifidobacterium breve, Bacteroides finegoldii, Bacteroides fragilis, Bacteroides massiliensis, Bacteroides ovatus, Bifidobacterium bifidum, Lactobacillus acidofilus, Lactobacillus casei, Lactobacillus reuteri, Streptococcus thermophilus, and Prevotella histicola.

24. The recombinant commensal bacterium of claim 23, wherein the bacterium is selected from the group consisting of Bacteroides thetaiotaomicron, Bacteroides vulgatus, Bacteroides finegoldii and Helicobacter hepaticus.

25. The recombinant commensal bacterium of any one of claims 22-24, wherein the disease or condition is an autoimmune disorder.

26. The recombinant commensal bacterium of claim 25, wherein the autoimmune disorder is selected from the group consisting of multiple sclerosis, diabetes mellitus Type I, rheumatoid arthritis, systemic lupus erythematosus, inflammatory bowel disease, celiac disease, Graves' disease, Hashimoto's autoimmune thyroiditis, vitiligo, rheumatic fever, pernicious anemia/atrophic gastritis, alopecia areata, immune thrombocytopenic purpura, temporal arteritis, ulcerative colitis, Crohn's disease, scleroderma, antiphospholipid syndrome, autoimmune hepatitis type 1, primary biliary cirrhosis, Sjogren's syndrome, Addison's disease, dermatitis herpetiformis, Kawasaki disease, sympathetic ophthalmia, HLA-B27 associated acute anterior uveitis, primary sclerosing cholangitis, discoid lupus erythematosus, polyarteritis nodosa, CREST Syndrome, myasthenia gravis, polymyositis/dermatomyositis, Still's disease, autoimmune hepatitis type 2, Wegener's granulomatosis, mixed Connective tissue disease, microscopic polyangiitis, autoimmune polyglandular syndrome, Felty's syndrome, autoimmune hemolytic anemia, chronic inflammatory demyelinating polyneuropathy, Guillain-Barre Syndrome, Behcet disease, autoimmune neutropenia, bullous pemphigoid, essential mixed cryoglobulinemia, linear morphea, autoimmune polyglandular syndrome 1 (APECED), acquired hemophilia A, Batten disease/neuronal ceroid lipofuscinoses, autoimmune pancreatitis, Hashimoto's encephalopathy, Goodpasture's disease, pemphigus vulgaris, autoimmune disseminated encephalomyelitis, relapsing polychondritis, Takayasu arteritis, Churg-Strauss syndrome, epidermolysis bullosa acquisita, cicatricial pemphigoid, pemphigus foliaceus, autoimmune hypoparathyroidism, autoimmune hypophysitis, autoimmune inner ear disease, autoimmune lymphoproliferative syndrome, autoimmune oophoritis, autoimmune orchitis, autoimmune polyglandular syndrome, Cogan's syndrome, encephalitis lethartica, erythema elevatum diutinum, Evans syndrome, immunodysregulation polyendocrinopathy enteropathy X-linked (IPEX), Issac's syndrome/acquired neuromyotonia, Miller Fisher syndrome, Morvan's syndrome, PANDAS, POEMS syndrome, Rasmussen's encephalitis, stiff-person syndrome, Vogt-Koyanagi-Harada syndrome, neuromyelitis optica, graft vs host disease, and autoimmune uveitis.

27. The recombinant commensal bacterium of claim 25 or 26, wherein the autoimmune disorder is selected from the group consisting of multiple sclerosis and diabetes mellitus Type I.

28. The recombinant commensal bacterium of claim 25 or 26, wherein the non-native protein or peptide is selected from the group consisting of ovalbumin, myelin oligodendrocyte glycoprotein, insulin, chromogranin A, hybrid insulin peptides, proteolipid protein, myelin basic protein, villin, epithelial cellular adhesion molecule, collagen alpha-1, aggrecan core protein, 60 kDa chaperonin 2, vimentin, alpha-enolase, fibrinogen alpha chain, fibrinogen beta chain, chitinase-3-like protein, 60 kDa mitochondrial heat shock protein, matrix metalloproteinase-16, thyroid peroxidase, thyrotropin receptor, thyroglobulin, gluten, TSHR protein, glutamate decarboxylase 2, receptor-type tyrosine-protein phosphatase-like N, glucose-6-phosphatase 2, insulin isoform 2, zinc transporter 8, glutamate decarboxylase 1, GAD65, UniProt:A2RGMO, integrin alpha-Iib, integrin beta-3, EBV DNA polymerase catalytic subunit, 2′3′-cyclic-nucleotide 3′ phosphodiesterase, myelin associated oligodendrocyte basic protein, small nuclear ribonucleoprotein, U1 small nuclear ribonucleoprotein, histone H2B, histone H2A, histone H3.2, beta-2-glycoprotein, histone H4, 60S ribosomal protein L7, TNF-alpha, myeloperoxidase, Cbir1, MS4A12, DNA topoisomerase, CYP2D6, O-phosphoseryl-tRNA selenium transferase, pyruvate dehydrogenase complex, spectrin alpha chain, steroid 21-hydroxylase, acetylcholine receptor, MMP-16, keratin associated proteins. Chondroitin sulfate proteoglycan 4, myeloblastin, U1 small nuclear ribonucleoprotein 70 kDa, blood group Rh(D), blood group Rh(CE), myelin P2 protein, peripheral myelin protein 22, myelin protein P0, S-arrestin, collagen Alpha-1, coagulation factor VIII, collagen alpha-3(IV), desmoglein-3, desmoglein-1, Insulin-2, major DNA-binding protein, tyrosinase, 5,6-dihydroxyindole-2-carboxylic acid oxidase, HLA-A2, aquaporin-4, myelin proteolipid protein, ABC transporter, HLA I B-27 alpha chain, HLA I B-7 alpha chain, and retinol-binding protein 3.

29. The recombinant commensal bacterial of claim 28, wherein the non-native protein or peptide is selected from the group consisting of ovalbumin, myelin oligodendrocyte glycoprotein, insulin, chromogranin A, hybrid insulin peptides, proteolipid protein, myelin basic protein, villin, epithelial cellular adhesion molecule,

30. The recombinant commensal bacterium of any one of claims 22-29, wherein the bacterium is engineered to secrete the expressed protein or peptide.

31. The recombinant commensal bacterium of any one of claims 22-29, wherein the bacterium is engineered to express a fusion protein comprising the protein or peptide and a native bacterial protein or portion thereof.

32. The recombinant commensal bacterium of claim 31, wherein the protein or peptide is fused to the N-terminus or the C-terminus of the native bacterial protein or portion thereof.

33. The recombinant commensal bacterium of claim 31 or 32, wherein the native bacterial protein is selected from the group consisting of sialidase, endonuclease, secreted endoglycosidase, anti-sigma factor, thiol peroxidase, hypothetical protein BT_2621, hypothetical protein BT_3223, peptidase, Icc family phosphohydrolase, exo-poly-alpha-D-galacturonosidase, and hypothetical protein BT_4428.

34. The recombinant commensal bacterium of claim 33, wherein the native bacterial protein is sialidase or anti-sigma factor.

35. The recombinant commensal bacterium of any one of claims 1-11, wherein the adaptive immune response is a Teffector response and the bacterium is selected from the group consisting of S. epidermidis, Corynebacterium spp., Parabacteroides distasonis, Parabacteroides gordonii, Alistipes senegalensis, Parabacteroides johnsonii, Paraprevotella xylaniphila, Bacteroides dorei, Bacteroides uniformis JCM 5828, Eubacterium limosum, Ruminococcaceae bacterium cv2, Phascolarctobacterium faecium, Fusobacterium ulcerans, Klebsiella pneumoniae, Clostridium bolteae 90B3, Clostridium cf. saccharolyticum K10, Clostridium symbiosum WAL-14673, Clostridium hathewayi 12489931, Ruminococcus obeum A2-162, Ruminococcus gnavus AGR2154, Butyrate-producing bacterium SSC/2, Clostridium sp. ASF356, Coprobacillus sp. D6 cont1.1, Eubacterium sp. 3_1_31 cont1.1, Erysipelotrichaceae bacterium 21_3, Subdoligranulum sp. 4_3_54A2FAA, Ruminococcus bromii L2-63, Firmicutes bacterium ASF500, Firmicutes bacterium ASF500, Bacteroides dorei 5_1_36/D4 supercont2.3, Bifidobacterium animalis subsp. Lactis ATCC 27673, and Bifidobacterium breve UCC2003.

36. The recombinant commensal bacterium of claim 35, wherein the bacterium is selected from the group consisting of S. epidermidis LM087 and Corynebacterium spp.

37. The recombinant commensal bacterium of claim 35 or 36, wherein the disease or condition is a proliferative disorder.

38. The recombinant commensal bacterium of claim 37, wherein the proliferative disorder is cancer.

39. The recombinant commensal bacterium of claim 38, wherein the cancer is selected from melanoma, basal cell carcinoma, squamous cell carcinoma, and testicular cancer.

40. The recombinant commensal bacterium of claim 38, wherein the cancer is melanoma.

41. The recombinant commensal bacterium of any one of claims 35-37 wherein the non-native protein or peptide is selected from the group consisting of PMEL, TRP2, MART-1, NY-ESO, MAGE-A, and a neoantigen.

42. The recombinant commensal bacterium of claim 41, wherein the non-native protein or peptide is PMEL.

43. The recombinant commensal bacterium of any one of claims 35-42, wherein the bacterium is engineered to secrete the expressed protein or peptide.

44. The recombinant commensal bacterium of any one of claims 35-42, wherein the bacterium is engineered to express a fusion protein comprising the protein or peptide and a native bacterial protein or portion thereof.

45. The recombinant commensal bacterium of claim 44, wherein the protein or peptide is fused to the N-terminus or the C-terminus of the native bacterial protein or portion thereof.

46. The recombinant commensal bacterium of any one of claims 35-45, wherein the native bacterial protein is selected from the group consisting of sialidase, endonuclease, secreted endoglycosidase, anti-sigma factor, thiol peroxidase, hypothetical protein BT_2621, hypothetical protein BT_3223, peptidase, Icc family phosphohydrolase, exo-poly-alpha-D-galacturonosidase, and hypothetical protein BT_4428.

47. The recombinant commensal bacterium of claim 46, wherein the native bacterial protein is sialidase or anti-sigma factor.

48. The recombinant commensal bacterium of any one of claims 1-47, wherein the bacterium is administered in combination with a high-complexity defined microbial community.

49. The recombinant commensal bacterium of any one of claims 1-48, wherein the host is a mammal.

50. The recombinant commensal bacterium of claim 49, wherein the mammal is a human.

51. A polynucleotide used to engineer the recombinant commensal bacterium of any one of claims 1-50.

52. A method for generating an antigen-presenting cell displaying an antigen derived from a non-native protein or peptide, comprising: administering the recombinant commensal bacterium of any one of claims 1-50 to a subject, wherein the administration results in colonization of the native host niche by the bacterium, internalization of the bacterium by an antigen-presenting cell, and presentation of the antigen by the antigen-presenting cell.

53. The method of claim 52, wherein the colonization of the native host niche is persistent or transient.

54. The method of claim 53, wherein the native host niche is transiently colonized, and wherein colonization is for 1 day to 60 days.

55. The method of claim 54, wherein the native host niche is transiently colonized, and wherein colonization is for 3.5 days to 60 days.

56. The method of claim 54 or 55, wherein the native host niche is transiently colonized, and wherein colonization is for 7 days to 28 days.

57. The method of any one of claims 52-56, wherein colonization is determined by polymerase chain reaction or colony forming assay performed on a sample obtained from the host after 1 day, 3.5 days, 7 days, 14 days, 28 days, or 60 days after administration to the host.

58. The method of any one of claims 52-57, wherein the administration results in interaction of the bacterium with a native immune system partner cell.

59. The method of claim 58, wherein the native immune system partner cell is the antigen-presenting cell.

60. The method of claim 59, wherein the antigen-presenting cell is selected from the group consisting of a dendritic cell, a macrophage, a B-Cell, and an intestinal epithelial cell.

61. The method of any one of claims 52-60, wherein the native host niche is selected from the group consisting of the gastrointestinal tract, respiratory tract, urogenital tract, and skin.

62. The method of any one of claims 52-61, wherein the presentation is within an MHC II complex.

63. The method of any one of claims 52-61, wherein the presentation is within an MHC I complex.

64. The method of any one of claims 52-63, wherein the bacterium is a Gram-negative bacterium.

65. The method of claim 64, wherein the Gram-negative bacterium is selected from the group consisting of Bacteroides thetaiotaomicron, Helicobacter hepaticus, Parabacteroides sp., and Prevotella spp.

66. The method of any one of claims 52-63, wherein the bacterium is a Gram-positive bacterium.

67. The method of claim 66, wherein the Gram-positive bacterium is selected from the group consisting of Staphylococcus epidermidis, Faecalibacterium sp. and Clostridium sp.

68. The method of any one of claims 52-63, wherein the administration is via a route selected from the group consisting of topical, enteral, parenteral and inhalation.

69. The method of claim 68, wherein the route is topical.

70. The method of claim 69, wherein the bacterium is S. epidermidis.

71. The method of claim 68, wherein the route is enteral.

72. The method of claim 71, wherein the bacterium is selected from the group consisting of Bacteroides spp., Clostridium spp., Helicobacter spp., Parabacteroides spp, and Prevotella spp.

73. The method of claim 72, wherein the bacterium is selected from the group consisting of Bacteroides thetaiotaomicron, Bacteroides vulgatus and Bacteroides finegoldii.

74. The method of claim 73, wherein the native bacterial protein is selected from the group consisting of sialidase, endonuclease, secreted endoglycosidase, anti-sigma factor, thiol peroxidase, hypothetical protein BT_2621, hypothetical protein BT_3223, peptidase, Icc family phosphohydrolase, exo-poly-alpha-D-galacturonosidase, and hypothetical protein BT_4428.

75. The method of claim 74, wherein the native bacterial protein is sialidase or anti-sigma factor.

76. The method of claim 75, wherein the non-native protein or peptide is melanocyte oligodendrocyte glycoprotein.

77. The method of claim 76, wherein the disease or condition is multiple sclerosis.

78. A method for generating a T-cell response in a subject, comprising: administering the recombinant commensal bacterium of any one of claims 1-50 to the subject, wherein the administration results in colonization of a native host niche by the bacterium and generation of the T-cell response, wherein the T-cell response is to an antigen derived from the non-native protein or peptide.

79. The method of claim 78, wherein the colonization of the native host niche is persistent or transient.

80. The method of claim 79, wherein the native host niche is transiently colonized, and wherein colonization is for 1 day to 60 days.

81. The method of claim 80, wherein the native host niche is transiently colonized, and wherein colonization is for 3.5 days to 60 days.

82. The method of claim 80 or 81, wherein the native host niche is transiently colonized, and wherein colonization is for 7 days to 28 days.

83. The method of any one of claims 78-82, wherein colonization is determined by polymerase chain reaction or colony forming assay performed on a sample obtained from the host after 1 day, 3.5 days, 7 days, 14 days, 28 days, or 60 days after administration to the host.

84. The method of any one of claims 78-83, wherein the administration is via a route selected from the group consisting of topical, enteral, parenteral and inhalation.

85. The method of claim 84, wherein the route is topical.

86. The method of claim 84, wherein the route is enteral.

87. The method of any one of claims 84-86, wherein the bacterium is selected from the group consisting of Bacteroides spp., Clostridium spp., Helicobacter spp., Parabacteroides spp, Prevotella spp., and Staphylococcus epidermidis.

88. The method of any one of claims 84-87, wherein the T-cell response is a Treg or a Teffector response.

89. The method of claim 88, wherein the route is enteral and the T-cell response is a Treg response.

90. The method of claim 89, wherein the bacterium is selected from the group consisting of Bacteroides spp., Clostridium spp., Helicobacter spp., Parabacteroides spp, and Prevotella spp.

91. The method of claim 90, wherein the bacterium is selected from the group consisting of Bacteroides thetaiotaomicron, Bacteroides vulgatus, and Bacteroides finegoldii.

92. The method of claim 91, wherein the native bacterial protein is selected from the group consisting of sialidase, endonuclease, secreted endoglycosidase, anti-sigma factor, thiol peroxidase, hypothetical protein BT_2621, hypothetical protein BT_3223, peptidase, Icc family phosphohydrolase, exo-poly-alpha-D-galacturonosidase, and hypothetical protein BT_4428.

93. The method of claim 92, wherein the native bacterial protein is sialidase or anti-sigma factor.

94. The method of claim 93, wherein the non-native protein or peptide is myelin oligodendrocyte glycoprotein.

95. The method of claim 94, wherein the disease or condition is multiple sclerosis.

96. The method of claim 88, wherein the route is topical and the T-cell response is a Teffector response.

97. The method of claim 96, wherein the bacterium is S. epidermidis.

98. A method of treating a disease or condition in a subject, comprising: administering the recombinant commensal bacterium of any one of claims 1-50 to the subject, wherein the administration results in colonization of a native host niche by the bacterium and generation of a T-cell response, wherein the T-cell response is to an antigen derived from the non-native protein or peptide, and wherein the T-cell response treats the disease or condition in the subject.

99. The method of claim 98, wherein the colonization of the native host niche is persistent or transient.

100. The method of claim 99, wherein the native host niche is transiently colonized, and wherein colonization is for 1 day to 60 days.

101. The method of claim 100, wherein the native host niche is transiently colonized, and wherein colonization is for 3.5 days to 60 days.

102. The method of claim 100 or 101, wherein the native host niche is transiently colonized, and wherein colonization is for 7 days to 28 days.

103. The method of any one of claims 98-102, wherein colonization is determined by polymerase chain reaction or colony forming assay performed on a sample obtained from the host after 1 day, 3.5 days, 7 days, 14 days, 28 days, or 60 days after administration to the host.

104. The method of claim any one of claims 98-104, wherein the disease or condition is selected from the group consisting of an autoimmune disorder and a proliferative disorder.

105. The method of claim 104, wherein the autoimmune disorder is selected from the group consisting of multiple sclerosis, diabetes mellitus Type I, rheumatoid arthritis, systemic lupus erythematosus, inflammatory bowel disease, celiac disease, Graves' disease, Hashimoto's autoimmune thyroiditis, vitiligo, rheumatic fever, pernicious anemia/atrophic gastritis, alopecia areata, immune thrombocytopenic purpura, temporal arteritis, ulcerative colitis, Crohn's disease, scleroderma, antiphospholipid syndrome, autoimmune hepatitis type 1, primary biliary cirrhosis, Sjogren's syndrome, Addison's disease, dermatitis herpetiformis, Kawasaki disease, sympathetic ophthalmia, HLA-B27 associated acute anterior uveitis, primary sclerosing cholangitis, discoid lupus erythematosus, polyarteritis nodosa, CREST Syndrome, myasthenia gravis, polymyositis/dermatomyositis, Still's disease, autoimmune hepatitis type 2, Wegener's granulomatosis, mixed Connective tissue disease, microscopic polyangiitis, autoimmune polyglandular syndrome, Felty's syndrome, autoimmune hemolytic anemia, chronic inflammatory demyelinating polyneuropathy, Guillain-Barre Syndrome, Behcet disease, autoimmune neutropenia, bullous pemphigoid, essential mixed cryoglobulinemia, linear morphea, autoimmune polyglandular syndrome 1 (APECED), acquired hemophilia A, Batten disease/neuronal ceroid lipofuscinoses, autoimmune pancreatitis, Hashimoto's encephalopathy, Goodpasture's disease, pemphigus vulgaris, autoimmune disseminated encephalomyelitis, relapsing polychondritis, Takayasu arteritis, Churg-Strauss syndrome, epidermolysis bullosa acquisita, cicatricial pemphigoid, pemphigus foliaceus, autoimmune hypoparathyroidism, autoimmune hypophysitis, autoimmune inner ear disease, autoimmune lymphoproliferative syndrome, autoimmune oophoritis, autoimmune orchitis, autoimmune polyglandular syndrome, Cogan's syndrome, encephalitis lethartica, erythema elevatum diutinum, Evans syndrome, immunodysregulation polyendocrinopathy enteropathy X-linked (IPEX), Issac's syndrome/acquired neuromyotonia, Miller Fisher syndrome, Morvan's syndrome, PANDAS, POEMS syndrome, Rasmussen's encephalitis, stiff-person syndrome, Vogt-Koyanagi-Harada syndrome, neuromyelitis optica, graft vs host disease, and autoimmune uveitis.

106. The method of claim 105, wherein the autoimmune disorder is selected from the group consisting of multiple sclerosis, and diabetes mellitus Type I.

107. The method of claim 104, wherein the proliferative disorder is cancer.

108. The method of claim 107, wherein the cancer is selected from melanoma, basal cell carcinoma, squamous cell carcinoma, and testicular cancer.

109. The method of claim 107, wherein the cancer is melanoma.

110. The method of any one of claims 98-109, wherein the administration is via a route selected from the group consisting of topical, enteral, parenteral and inhalation.

111. The method of claim 110, wherein the route is topical.

112. The method of claim 111, wherein the bacterium is S. epidermidis.

113. The method of claim 112, wherein the disease is cancer.

114. The method of claim 113, wherein the cancer is melanoma.

115. The method of claim 113, wherein the non-native protein or peptide is selected from the group consisting of a melanocyte-specific antigen and a testis cancer antigen.

116. The method of claim 115, wherein the melanocyte-specific antigen is selected from the group consisting of PMEL, TRP2 and MART-1.

117. The method of claim 115, wherein the testis cancer antigen is selected from the group consisting of NY-ESO and MAGE-A.

118. The method of claim 110, wherein the route is enteral.

119. The method of claim 118, wherein the bacterium is selected from the group consisting of Bacteroides spp., Clostridium spp., Helicobacter spp., Parabacteroides spp, and Prevotella spp.

120. The method of claim 119, wherein the bacterium is selected from the group consisting of Bacteroides thetaiotaomicron, Bacteroides vulgatus, and Bacteroides finegoldii.

121. The method of claim 120, wherein the native bacterial protein is selected from the group consisting of sialidase, endonuclease, secreted endoglycosidase, anti-sigma factor, thiol peroxidase, hypothetical protein BT_2621, hypothetical protein BT_3223, peptidase, Icc family phosphohydrolase, exo-poly-alpha-D-galacturonosidase, and hypothetical protein BT_4428.

122. The method of claim 121, wherein the native bacterial protein is sialidase or anti-sigma factor.

123. The method of any one of claims 118-122, wherein the autoimmune disorder is selected from the group consisting of multiple sclerosis, diabetes mellitus Type I, rheumatoid arthritis, systemic lupus erythematosus, inflammatory bowel disease, celiac disease, Graves' disease, Hashimoto's autoimmune thyroiditis, vitiligo, rheumatic fever, pernicious anemia/atrophic gastritis, alopecia areata, immune thrombocytopenic purpura, temporal arteritis, ulcerative colitis, Crohn's disease, scleroderma, antiphospholipid syndrome, autoimmune hepatitis type 1, primary biliary cirrhosis, Sjogren's syndrome, Addison's disease, dermatitis herpetiformis, Kawasaki disease, sympathetic ophthalmia, HLA-B27 associated acute anterior uveitis, primary sclerosing cholangitis, discoid lupus erythematosus, polyarteritis nodosa, CREST Syndrome, myasthenia gravis, polymyositis/dermatomyositis, Still's disease, autoimmune hepatitis type 2, Wegener's granulomatosis, mixed Connective tissue disease, microscopic polyangiitis, autoimmune polyglandular syndrome, Felty's syndrome, autoimmune hemolytic anemia, chronic inflammatory demyelinating polyneuropathy, Guillain-Barre Syndrome, Behcet disease, autoimmune neutropenia, bullous pemphigoid, essential mixed cryoglobulinemia, linear morphea, autoimmune polyglandular syndrome 1 (APECED), acquired hemophilia A, Batten disease/neuronal ceroid lipofuscinoses, autoimmune pancreatitis, Hashimoto's encephalopathy, Goodpasture's disease, pemphigus vulgaris, autoimmune disseminated encephalomyelitis, relapsing polychondritis, Takayasu arteritis, Churg-Strauss syndrome, epidermolysis bullosa acquisita, cicatricial pemphigoid, pemphigus foliaceus, autoimmune hypoparathyroidism, autoimmune hypophysitis, autoimmune inner ear disease, autoimmune lymphoproliferative syndrome, autoimmune oophoritis, autoimmune orchitis, autoimmune polyglandular syndrome, Cogan's syndrome, encephalitis lethartica, erythema elevatum diutinum, Evans syndrome, immunodysregulation polyendocrinopathy enteropathy X-linked (IPEX), Issac's syndrome/acquired neuromyotonia, Miller Fisher syndrome, Morvan's syndrome, PANDAS, POEMS syndrome, Rasmussen's encephalitis, stiff-person syndrome, Vogt-Koyanagi-Harada syndrome, neuromyelitis optica, graft vs host disease, and autoimmune uveitis.

124. The method of claim 123, wherein the autoimmune disorder is multiple sclerosis.

125. The method of claim 124, wherein the bacterium is selected from the group consisting of Bacteroides thetaiotaomicron, Bacteroides vulgatus, and Bacteroides finegoldii.

126. The method of claim 125, wherein the non-native protein is myelin oligodendrocyte glycoprotein.

127. The method of any one of claims 52-126, wherein the bacterium is administered in combination with a high-complexity defined microbial community.

128. The method of any one of claims 52-127, wherein the host is a mammal.

129. The method of claim 128, wherein the mammal is a human.
